(19) United States
(12) Patent Application Publication
(10) Pub. No.: US 2009/0137641 A1
(43) Pub. Date: May 28, 2009

(54) Potassium Channel Activators for the Prevention and Treatment of Dystonia and Dystonia-Like Symptoms

(76) Inventors: Chris Rundfeldt, Coswig (DE); Angelika Richter, Braunschweig (DE)

Correspondence Address: Beusse Wolter Sanks Mora & Maire, 390 N. ORANGE AVENUE, SUITE 2500, ORLANDO, FL, 32801, US

(21) Appl. No.: 12/296,960

(22) PCT Filed: Apr. 30, 2007
(86) PCT No.: PCT/EP2007/003830

§ 371 Date: Oct. 13, 2008

(30) Foreign Application Priority Data
May 2, 2006 (EP) 06009064.4

(51) Int. Cl. A61K 31/44; A61K 31/24; A61K 31/404

(52) U.S. Cl. 514/353; 514/535; 514/418

(57) ABSTRACT

The present invention is directed to the prevention, reversal and medical treatment of dystonia and dyskinesia as well as other diseases related to movement disorders, both in human beings and animals by administering a neuronal potassium channel opener such as flupirtine, retigabine or maxipost.

### 1. FIELD OF THE INVENTION

The present invention is directed to the prevention, reversal and medical treatment of dystonia, dystonic symptoms and dystonia-associated dyskinesias, both in human beings and animals.

### 2. BACKGROUND INFORMATION

2.1. Classification of dystonia and dystonic symptoms

Dystonia is a neurological syndrome characterized by sustained, sometimes painful, muscle contractions that frequently cause twisting or repetitive movements and abnormal postures. Dystonia may affect any part of the body including the arms and legs, trunk, neck, head, or face. This disorder can involve any voluntary muscle in the body. Dystonia is an often intractable movement disorder which is frequently misdiagnosed (Fahn et al. 1998; Saunders-Pullman and Bressman, 2005). Dystonia is generally classified according to the age of onset, the distribution of symptoms and the etiology. The symptoms of dystonia may begin during childhood (i.e., early onset), adolescence, or adulthood. Age at onset is an important prognostic indicator. Generally, the earlier the onset of symptoms, the more likely the spread to other body parts (Greene et al., 1995). For example, the symptoms of generalized dystonia or dopa-responsive dystonia often begin during childhood, while focal dystonias usually occur in adolescence. Most cases of early-onset dystonia are thought to occur as the result of an inherited defect in a gene. Other cases may result from a spontaneous change in a gene. Certain focal dystonias such as cervical dystonia (spasmodic torticbllis), blepharospasm, writers cramp, and spasmodic dysphonia are examples of late-onset dystonias.

The classification of dystonia according to the distribution of symptoms includes: focal dystonia, segmental dystonia, multifocal and generalized dystonia (Jankovic and Fahn, 1998). Symptoms may be focal or limited to one region of the body, such as the neck or an arm or a leg. There are many different types of focal dystonia. Blepharospasm is marked by involuntary contraction of the muscles that control the movement of the eyelids. Symptoms may range from intermittent, painless, increased blinking to constant, painful, eye closure leading to functional blindness. In patients with cervical dystonia, also known as spasmodic torticollis, muscle spasms of the head and neck may be painful and cause the neck to twist into unusual positions or postures. These sometimes painful spasms may be intermittent or constant. Oromandibular and lingual dystonia are characterized by forceful contractions of the lower face causing the mouth to open or close. Chewing and unusual tongue movements may also occur. In spasmodic dysphonia, also known as laryngeal dystonia, the muscles in the larynx are affected. This leads to changes of the voice (hoarse, strangled or whispering quality). In limb dystonia, there are involuntary contractions of one or more muscles in the arm, hand, leg, or foot. These types of focal dystonias include writer's cramp and other occupational dystonias.

Some patients have symptoms that are segmental or involve two adjacent areas of the body, such as the head and neck or arm and trunk. In other patients, symptoms may be multifocal or appear in two areas of the body that are not next to each other, such as the two arms, or an arm and a leg. In generalized dystonia, symptoms begin in an arm or a leg and progress, becoming more widespread. Eventually, the trunk and the rest of the body are involved.

In view of the etiology, dystonias are divided into idiopathic or primary (unknown etiology) and symptomatic or secondary (dystonia as a symptom) types. The etiologies of dystonias and of dystonic symptoms are broad. Often the real cause remains unclear. Most cases of idiopathic dystonia are believed to be hereditary and to occur as the result of a faulty gene(s). The term primary dystonia is preferred for types of idiopathic dystonias in which abnormal genes have been discovered as the cause (Fahn et al., 1998). For example, most cases of early-onset primary dystonia (so-called primary torsion dystonia) are due to a mutation in the DYT-1 gene. Early-onset dystonia that occurs as a result of this disease gene is the most common and severe type of hereditary dystonia. In these patients, dystonia usually occurs as a solitary symptom and is not associated with an underlying disorder. Other genetic causes of primary dystonia are rare (Saunders-Pullman and Bressman, 2005).

Secondary dystonia may result from certain environmental factors or “insults” that affect the brain. It can occur as a symptom of another underlying disease such as Wilson disease, multiple sclerosis or may be caused by insults such as stroke and injury of the nervous system (e.g., by lack of oxygen during birth or as a result of an injury during a vehicular accident) or by drugs as a side effect (Jankovic and Fahn, 1998; Saunders-Pullman and Bressman, 2005). In adults, the most common type of secondary dystonia includes drug-induced dyskinesias (dystonia and other motor disturbances). Tardive dystonia is a type of persistent neuropeptic-induced dystonia. Drugs which may cause such tardive dystonia are certain neuroleptic or antipsychotic drugs (used to treat psychiatric disorders). These drugs include but are not limited to haloperidol or chlorpromazine. Other drugs that block central dopamine receptors, i.e. dopamine receptor antagonists, may also cause tardive dystonia. The most common forms of these dystonia like symptoms are called dyskinesia. Otherwise, dopaminergic drugs such as levodopa and dopamine receptor agonists can provoke dyskinesias (including dystonia) in patients with Parkinson's disease (Fahn et al., 1998). In most patients, symptoms occur some time after ongoing exposure to the drug. Interestingly, symptoms may continue even after cessation of treatment.

Dystonia may be associated with certain non-degenerative, neurochemical disorders (known as “dystonia-plus syndromes”) that are characterized by other neurologic features, such as parkinsonism, and responds to levodopa (so-called dopa-responsive dystonia). Dystonia is also a primary feature of certain, usually hereditary, neurodegenerative disorders (so-called “heredodegenerative dystonias”). However, in some patients with these disorders, dystonia may not develop and other neurologic features may be primary findings. The term “heredodegenerative” is used since many of these disorders are hereditary; however, it is important to note that some are of unknown cause. The heredodegenerative dystonias include numerous disorders, such as certain X-linked recessive, autosomal dominant, autosomal recessive, and/or parkinsonism syndromes. Such disorders include but are not limited to the following syndromes: X-linked dystonia-parkinsonism (Lubag), juvenile parkinsonism, Huntington's disease, Wilson's disease, neuroacanthocytosis, Rett syndrome, Parkinson's disease, other autosomal recessive disorders (such as ataxia-telangiectasia, Hallervorden-Spatz disease, and homocystinuria), certain mitochondrial disorders (e.g. Leigh disease), other parkinsonism disorders, including progressive supranuclear palsy and cortical-basal degeneration.

Dystonia may also occur in association with other episodic neurologic movement disorders, which include dystonic tics and paroxysmal dyskinesias. Paroxysmal dyskinesias are a group of episodic movement disorders that may include any combination of dystonia and other involuntary movements, such as chorea, athetosis, or balism. There are four major types of paroxysmal dyskinesias differentiated by the precipitating and exacerbating factors: paroxysmal non-kinesigenic dyskinesia (paroxysmal dystonic choreoathetosis; briefly: paroxysmal dystonia), paroxysmal kinesigenic dyskinesia (paroxysmal kinesigenic choreoathetosis), the exertion-induced and hypnogenic paroxysmal dyskinesias (Nardocci et al., 2002). For example, in patients with paroxysmal dystonia episodes of generalized dystonia (the predominant feature) last up to several hours and can be provoked by stress and caffeine.

Apart from paroxysmal dyskinesias, other types of dystonia (see above) are usually persistent, but can be worsened by stress and exercise. In drug-induced dyskinesias, dystonic symptoms often fluctuate in dependence to drug-intake. Furthermore, the so-called action dystonias can be exacerbated by specific movements, i.e., focal dystonias in musicians and writer's cramps (Jankovic and Fahn, 1998). Dystonia like symptoms occur also in patients with restless leg syndrome, in patients with Tourette's syndrome and in patients with Tics. Dystonia-like symptoms can be also observed in patients with neuromyotonia (also known as Isaacs' Syndrome), with myokymia, Restless legs syndrome, Stiff person syndrome, Multiple sclerosis and Central pontine myelinolysis.

As a result of muscular hyperactivity in neuromyotonia, patients may present with muscle cramps, myotonia-like symptoms, excessive sweating, myokymia and fasciculations. A very small proportion of cases with neuromyotonia may develop central nervous system findings in their clinical course, causing a disorder called Morvan's syndrome and they may also have antibodies against potassium channels in their serum samples.

### 2.2. Epidemiology

Despite the greater prevalence of dystonia than other well-known neurological conditions, such as myasthenia gravis and motor neuron disease, there are limited data on the frequency of dystonia (Saunders-Pullman and Bressmann, 2005). Due to the variability of associated symptoms and disease severity and the fact that some patients with mild cases may remain undiagnosed, it is difficult to determine the specific frequency of dystonia in the general population. Current prevalence estimates of dystonia range from 6.1 individuals per 100,000 for focal dystonia to 34 per 100,000 for all primary dystonias. There are few epidemiological studies on dystonia and its various forms. A large European study, reported in the literature in 2000, estimated the crude annual period prevalence rate for primary dystonia (for 1996-1997) at 152 per million. Of the primary dystonias, focal dystonia had the highest relative rate at 117 per million. The prevalence rates for the other dystonias were estimated as follows: 57 per million for cervical dystonia; 36 per million for blepharospasm; and 14 per million for writer's cramp. The relative rates, adjusted for age, were substantially higher in women than in men for the segmental and focal dystonias. The exception to this was writer's cramp. The authors point out that these limited data are most likely underestimated (Saunders-Pullman and Bressman, 2005).

### 2.3. Pathophysiology

By using standard techniques, no pathomorphological alterations could be detected within the CNS of patients with idiopathic dystonias, while symptomatic types are often associated with lesions in basal ganglia nuclei, particularly the striatum (Bhatia and Marsden, 1994). No consistent or specific changes in brain tissue or function have been seen in individuals with primary dystonias, and the basic underlying defect or defects in these disorders remain unknown. It has been suggested that idiopathic dystonias probably result from abnormalities in the activity of neurotransmitters, such as an imbalance of dopamine transmission, within the basal ganglia. This hypothesis is based on pharmacological observations, but the significance of dopamine in the pathogenesis of dystonia remains uncertain, except for dopa-responsive dystonia. An underlying neurochemical basis for many dystonias may be suggested by multiple factors, including evidence that secondary dystonia may result from treatment with the dopamine precursor L-dopa (such as used for treatment of Parkinson's disease) or therapy with dopamine receptor blockers (antagonists). As mentioned earlier, the dystonia-plus syndromes also are nondegenerative, neurochemical disorders that are distinguished from primary dystonias due to the presence of neurologic features in addition to dystonia (e.g., myoclonus or parkinsonism). Specifically, dopa-responsive dystonia (DRD) and several DRD variants have been shown to result from reduced production of dopamine and/or other neurotransmitters in the basal ganglia.

Abnormalities in the activities of certain neurotransmitters have also been demonstrated in heredodegenerative disorders (e.g., Parkinson's disease, Rett syndrome, and others). In addition, anatomic studies of focal brain lesions associated with certain secondary dystonias and specific neurodegenerative changes found in heredodegenerative dystonias (e.g., Wilson's disease, Huntington's disease, neuroacanthocytosis, etc.) implicate dysfunction of the basal ganglia and its connections (e.g., thalamus, cerebral cortex, or, rarely, the brainstem) as a cause of such dystonias—and further support the theory that primary dystonias may result from abnormalities of the basal ganglia.

Although the pathophysiology is probably heterogeneous dependent on the type of dystonia, there is evidence that different types of dystonia are related to an abnormal activity of specific neurons within basal ganglia nuclei which control motor functions (Wichmann and DeLong, 1996; Vitek and Giroux, M., 2000).

### 2.4. Treatment

There are three main approaches to the treatment of dystonia: oral medications, injections of therapeutic agents directly into dystonic muscle in patients with focal dystonias, and surgery in patients which do not benefit from medical treatment. Physical therapy may play a role as a supplement to medical treatment. The first step in treatment is attempting to determine the cause of the dystonia, because for secondary dystonias, treating the underlying cause may improve the dystonia. In most cases of dystonia, the treatment is merely symptomatic, designed to improve posture, motor function and to relieve associated pain (Jankovic, 2004). For instance, treatments for neurological conditions such as multiple sclerosis or Parkinson's disease may reduce dystonic symptoms. Withdrawing or reducing neuroleptic drugs leads to slow improvement in some cases. Interestingly, while neuroleptic treatment may be the cause of dystonia, a withdrawal of this medication does often not lead to a full remission indicating that adaptive changes resulting from neuroleptic treatment may result in dystonia. There are currently no known treatments that can reverse the course of idiopathic dystonias. However, symptoms may usually be managed to a certain extent with a combination of treatments, however often at the expense of drug related side effects. The selection of a particular choice of treatment is largely guided by empirical trials. The response to drugs is often disappointing and depends on the type of dystonia (Fahn, 1995).

For example, patients with dopa-responsive dystonia (DRD) improve significantly with small doses of levodopa. Therefore, neurologists often try a course of levodopa therapy for patients with generalized dystonia in order to determine if DRD is the cause. Most patients with other types of dystonia do not benefit from levodopa or to other dopaminergic drugs, such as dopamine agonists.

Focal dystonias are often successfully treated with botulinum toxin. Botulinum toxin (BTX) is a biological therapeutic agent that acts against focal dystonia, when a minute amount of commercially prepared toxin is injected directly into an overactive muscle. This treatment, relaxes the muscle for several months.

Apart from DRD and focal dystonias, medical treatments are however often disappointing (Fahn, 1995). Benzodiazepines are a class of drugs that interfere with chemical activities in the nervous system and brain, serving to reduce communication between nerve cells. Consequently, such medications may relax muscles and ease symptoms associated with dystonia. Benzodiazepines are oral medications that may be used to treat focal, segmental, and generalized dystonias. Diazepam and clonazepam are two types of benzodiazepines that are most commonly used to treat dystonia. The major side effect of these drugs is drowsiness, which may be controlled by lowering the dose. At relatively high doses, side effects may include depression, personality changes, or, in severe cases, psychosis. These drugs also have a high addictive potential and due to development of tolerance the treatment effect may be lost upon long term treatment. Baclofen is a drug that is used to treat individuals with spasticity. In addition, this drug has been administered to some patients with dystonia. Baclofen's primary site of action is the spinal cord where it reduces the release of neurotransmitters that stimulate muscle activity by stimulating GABA_{B} autoreceptors. Baclofen has been used to treat both primary and secondary dystonias. This drug may be administered orally or via a surgically implanted pump that delivers the drug directly to the spinal cord (intrathecal baclofen). Anticholinergic drugs block the action of the neurotransmitter acetylcholine, thereby deactivating muscle contractions. These drugs are administered orally and used to treat focal, segmental, and generalized dystonias. Trihexyphenidyl and diphenhydramine are the most common anticholinergic agents used to treat dystonia (diphenhydramine is also an anti-histaminic drug). This form of therapy may be more beneficial in children, as they are frequently able to tolerate higher doses of trihexyphenidyl than adults. Greater therapeutic benefits may also occur in those patients who initiate drug therapy early during the course of their disease. Side effects may be severe, particularly at higher doses. These may include confusion, drowsiness, hallucinations, forgetfulness, personality changes, dry mouth, blurred vision, and urinary retention. Dopamine-blocking or dopamine-depleting agents may be used to treat some patients with dystonia. The possible positive effect of these agents is a paradox since dopamine blockers may also cause dystonia. Nonetheless, these agents have been shown to be effective in some patients. Tetrabenazine is the most widely used dopamine-blocking agent. In some patients, tetrabenazine may be combined with lithium, which may help to lessen side effects of tetrabenazine such as slowed movements and depression. Other dopamine blockers are not as commonly used, since they may be more likely to evoke tardive dystonia. The neuroleptic drugs clozapine and olanzapine may be useful for the treatment of dystonia and may be less likely to cause tardive dystonia.

### 2.5. Neuronal Potassium Channel Openers

Channels selective for K⁺ ions play a vital role in the function of many cell types (Rudy, 1988; Hille, 1993). They are exceptionally diversified both in variety and function. Individual cells can, and normally do, express several kinds of channels.

Such channels are regulated through various mechanisms (Rudy, 1988) and can be grouped into different gene families. Differences between K⁺ channels have also emerged clearly from molecular biology studies showing that they differ considerably in molecular structure (Takumi et al., 1988; Kubo et al., 1993; Ruppersberg et al., 1993). K⁺ channels are currently target of diverse pharmacological manipulation. Voltage activated K⁺ channels in the heart are blocked by class III antiarrhythmic drugs such as amiodarone and sotalol, and this action delays the repolarization of the cardiac action potential and increases cardiac refractoriness (Colatsky et al., 1990). The antidiabetic sulfonylureas glibenclamide and tolbutamide are blocker of the ATP-sensitive K⁺ channel, KATP, and these drugs affect insulin producing β-cells (Bernardi and Lazdunski, 1993). K⁺ channel openers such as levcromakalim, aprikalim and pinacidil, currently being evaluated for the treatment of hypertension, peripheral ischemia and obstructive airway diseases, also influence the KATP channel (Edwards and Weston, 1993). Modulators of a subtype of Ca²⁺ activated K⁺ channels, namely the large conductance Ca²⁺ activated K⁺ channels (BKₘₐₓ), are being evaluated for neuroprotective activity. Such Ca²⁺ activated K⁺ channels are expressed in most neurones, smooth and striated muscle cells and secretory epithelial cells (McKay et al., 1994). Inward rectifying K⁺ channels, which are open in resting cells, are involved in the generation of the resting membrane potential which is mainly due to the concentration gradient of K⁺ ions. While the whole cell conductance of such channels is fairly small, the contribution to cell membrane potential is large since the input resistance of neuronal cells is high and open channels do not desensitize. A selective opening of such channels is discussed as a therapeutic target for several diseases including epilepsy and neurodegeneration (Doupnik et al., 1995).

More recently, other families of K⁺ channels have gained also interest as therapeutic targets. The different members of the KCNQ channel family, recently renamed as Kv7 channel family, are differentially expressed in diverse tissues. While KCNQ1 (Kv7.1) is expressed in the heart muscle, KCNQ2, 3 and 4 subunits are predominately expressed in neuronal cells and are target of the anticonvulsant and analgesic retigabine (Rundfeldt and Netzer, 2000). KCNQ3 and 5 subunits are also expressed in bladder smooth muscle cells and may serve as a new target for the treatment of urinary incontinence. Again other potassium channels, namely the Kv1.3 channels as member of the Kv1 family is expressed among other tissues in immune cells and is discussed as target for immune modulation for the treatment of autoimmune diseases and chronic inflammation (Vennekamp et al. 2004), while a different member of the same family, the Kv1.5 channel, is a target for the treatment of cardiac arrhythmia. The role of openers for both these channels is not yet determined.

### 2.6 Conclusion

Dystonia can be an idiopathic disease, a symptom occurring in several inherited and/or degenerative disorders or a result of exogenous causes including of drug treatments, i.e., a side effect of treatment of certain diseases with pharmaceuticals. Despite the broad spectrum of available drugs to prevent, treat or ameliorate this movement disorder the treatment remains in many cases in-satisfactory and no causal treatment is available. Potassium channels are diverse and are discussed as targets for numerous diseases as they are involved in vital functions of different cell types. Due to the high diversity of potassium channels and due to the often organ specific distribution, potassium channel modulators bear the potential to be interesting drugs for numerous diseases. However, no data are available linking potassium channel modulation and especially modulation of KCNQ channels as well as modulation of Kir channels to the treatment of dystonia.

### 3. SUMMARY OF THE INVENTION

In an attempt to find better treatments for dystonia including neuroleptics induced dystonia and dystonia-associated dyskinesias which include levodopa-induced dyskinesia, neuromytononia, myokymia and other diseases resulting in dystonia like symptoms, we have tested activators of different potassium channels which are expressed in neuronal cells (i.e. neuronal potassium channel activators) in the dt^{sz} mutant hamster, a genetic animal model of paroxysmal dyskinesia in which dystonia is the predominant feature. This model was published previously and drugs which have been found to be effective in this model are also used for the treatment of dystonia in man (Richter and Löscher, 1998; Richter, 2005). In addition, the compounds were also tested in a model of L-DOPA induced dystonia (L-DOPA-induced dyskinesia). The model has been developed by Cenci, Lee and Bjorklund (L-DOPA-induced dyskinesia in the rat is associated with striatal overexpression of prodynorphin- and glutamic acid decarboxylase mRNA, Eur J. Neurosci. 1998; 10:2694-706) and was modified to show more severe dyskinesia.

Unexpectedly, activators of different neuronal potassium channels have been found to be very active in suppressing the symptoms of dystonia in these two predictive animal models of dystonia/dyskinesia. An activation of KCNQ channels using the anticonvulsant retigabine (Rundfeldt and Netzer, 2000) was found to exert beneficial effects. The activation of G-protein coupled inward rectifying potassium channels (Kir channels) by using the analgesic flupirtine (Jakob and Krieglstein, 1997; Kornhuber et al., 1999) was also found to be effective (see Exhibit 1 and 2). Stimulation of a large conductance calcium activated potassium channel, the BKmax channel, by using the activator maxipost, was also found to be active. While all three channel families are distinct, they lead to a common feature, i.e., they are all described to stabilize the membrane potential and to lead to hyperpolarization of neuronal cells. Thus, based on these data, it can be concluded, that a potassium channel activation leading to stabilization of the membrane potential and to hyperpolarization of the membrane potential in neuronal cells (i.e. activation of neuronal potassium channels) is a new strategy for the treatment of dystonia. Among these potassium channels investigated, the KCNQ channel family (renamed: Kv7 channel family) was further investigated. We used the selective Kᵥ7.2/7.3 channel blocker 10,10-bis(4-pyridinylmethyl)-9(10H)-anthracenone (XE-991) to evaluate the effect of an antagonist, preventing the activity of the agonist retigabine. Interestingly and unexpectedly, the KCNQ channel blocker, administered at the dose of 3 and 6 mg/kg i.p., aggravated the dystonic attacks observed in the hamsters. In addition, the anti-dystonic effect of retigabine was counteracted by pre-treatment with XE-991 (see Exhibit 2). This experiment further highlights the role of neuronal potassium channels for dystonia. Thus, these data further support the observation, that activation of neuronal potassium channels results in anti-dystonic effect. This symptomatic improvement with regard to the movement disorder is to be distinguished from other pharmacological effects. The well known analgetic of Kᵥ7.2/7.3 channel openers and also of flupirtine may add to the over all beneficial effect of such compounds since dystonia often is associated with musculoskeletal pain (Nielsen et al., 2004). This combination of effect, i.e. the anti-dystonic effect and the analgetic effect, is unique to these Kᵥ7 channel openers and is important to relive dystonia-induced painful muscle spasms.

### 4. DETAILED DESCRIPTION OF THE INVENTION

### 4.1. Chemicals Used as Model Activators of Different Neuronal Potassium Channels and as Potential Drugs to Treat Dystonia

### 4.1.1. Flupirtine

Flupirtine (ethyl-N-[2-amino-6-(4-fluorophenylmethylamino)pyridin-3-yl]-carbamate), a triaminopyridine compound with antinociceptive effects, is marketed in Germany and some other countries for the treatment of centrally mediated pain under the trademark Katadolon™. It is an analgesic that has been used in Europe to treat pain association with surgery, cancer, trauma, dental pain, degenerative rheumatic arthrosis, and inflammatory rheumatoid arthritis and liver disease. It acts via central nervous system through nonopiate pain pathways, possibly involving the thalamus or spinal pain pathways. In some, but not all, studies flupirtine has been found to be as effective as opiates in relieving pain. Moreover, flupirtine offers a clear advantage over opiates in that it is not addictive and there have been no reports of abuse. The drug is very well tolerated and is free of effects on the cardiovascular system in patients.

While early work identified different potential mechanisms of action, lately the attention was focussed on its ability to activate neuronal potassium channels, namely the inward rectifying potassium channel GIRK. This lead to the development of the concept that flupirtine is a SNEPCO (selective neuronal potassium channel opener; Kornhuber et al., 1999). Flupirtine was found to be active in the treatment of different diseases. Several patents have been issues dealing with the use of flupirtine. Early work focussed on the analgesic activity. This was lately extended to the use of flupirtine for the treatment of canine and feline arthritis (EP 1242078, entitled “VERWENDUNG VON FLUPIRTINE ZUR LINDERUNG VON SCHMERZEN BEI DEGENERATIVEN GELENKERKRANKUNGEN VON HUNDEN UND KATZEN”). Combined therapy with opioids was also claimed to further improve the analgesic activity (EP 0595311, entitled “Kombinationspraparat aus Flupirtine und Morphin zur Behandlung von Schmerzen und zur Vermeidung der Morphin-Abhangigkeit”).

Later on especially neuroprotective effects and cytoprotective effects were published in several patents, for example in DE 69429435 T2/EP 0716602, entitled “Primary and secondary neuroprotective effect of flupirtine in neurodegenerative diseases” or in DE 19625582 A1, entitled “Use of flupirtine for the prophylaxis and therapy of disorders which are associated with an unphysiologically high cell death rate” or in EP 0912177, entitled “VERWENDUNG VON FLUPIRTINE GEGEN ZELLSCHÄDEN DURCH APOPTOSE UND NEKROSE”. This was extended to other organ systems in EP 0912177, entitled “VERWENDUNG VON FLUPIRTINE ZUR THERAPIE UND PROPHYLAXE VON MYOKARDINFARKT, SCHOCKNIERE UND SCHOCKLUNGE. A different therapeutic target was defined to be the hamatopoetic system, i.e. in DE 19541405 A1/EP 0859613, entitled “Use of flupirtine for the prophylaxis and therapy of diseases associated with an impairment of the haematopoetic cell system”. Other diseases to be treated with flupirtine include DE 10048969 A1 “VERWENDUNG VON FLUPIRTINE ZUR TINNITUSBEHANDLUNG”, EP 0659410 “Pharmaceutical composition comprising flupirtine and its use to combat muscular tension”, WO 00/59487, “Flupirtine in the treatment of fibromyalgia and related conditions”, WO 01/39760, “Method of treating batten disease”, U.S. Pat. No. 5,284,861 “Pharmaceutical composition comprising flupirtine and its use to combat Parkinson disorders”. Flupirtine is described to be active in animal models of Parkinson's disease in that the drug alleviates the symptoms of Parkinson's disease and in that it potentiates the activity of the anti-parkinsonian drug L-DOPA. Furthermore, due to the neuroprotective effect of flupirtine, the drug is described to counteract the progression of Parkinson's disease (G. Schuster, M. Schwarz, F. Block, G. Pergande, and W. J. Schmidt, 1998: Flupirtine: A Review of Its Neuroprotective and Behavioral Properties. CNS Drug Reviews Vol. 4, No. 2, pp. 149-164). Furthermore, patents applications have been issued to describe different dosage forms, such as DE 10255415 A1, “Cutaneous application of flupirtine”, or EP 0615754, “Oral forms of administration containing solid flupirtine with controlled release of active substance”.

However, despite of the widespread use and examination of flupirtine, it has not previously been known to be useful for the treatment of dystonia and dystonia-associated dyskinesias with the aim to reduce the severity of dystonia and to relieve dystonia-related muscle pain. It is to be noted that in Parkinson's patients there is a high rate of L-DOPA-induced dystonia/dyskinesia. The symptoms of this drug-induced dystonia are clearly distinct from the underlying Parkinson's disease symptoms and treatment of the L-DOPA-induced dyskinesia is one of the unmet medical needs in Parkinson's disease patients. The present invention is based upon the discovery that flupirtine as one of the used model drugs for a neuronal potassium channel activator is unexpectedly effective in alleviating symptoms of dystonia including L-DOPA-induced dyskinesia and Neuroleptics-induced dyskinesia.

### 4.1.2. Retigabine

Retigabine, 2-amino-4-(4-fluorobenzylamino)-1-ethoxycarbonyl-aminobenzene, is a known anti-convulsant compound described for example in U.S. Pat. No. 5,384,330. Retigabine was identified as a selective activator of KCNQ2/3 potassium channels (see for example WO 01/01970). Other authors describe retigabine to be also active on KCNQ4 and KCNQ5 channels.

Retigabine and other KCNQ2/3 modulators are described as analgesics, fever reducers, muscle relaxants, anxiolytics and are of use in migraine, bipolar disorders and unipolar depression, tinnitus and in reducing dependence and drug addiction (WO 01/01970). Retigabine is also described to be active in reducing neuropathic pain. Effects or retigabine and its derivatives are also described in different patents (WO 01/10381, WO 01/22953, WO 02/00217, WO 02/032419, WO 02/49628, WO 02/72088, WO 02/80898) which are herein incorporated by reference.

### 4.1.3. Maxipost

Maxi post (BMS 204352), 3-(5-chloro-2-methoxyphenyl)-1,3-dihydro-3-fluoro-6-(trifluoromethyl)-2H-indole-2-one, as a racemate and also with regard to both enantiomers and its prodrugs, is a potent and effective opener of calcium activated large conductance potassium channels (BKmax) and its actions are highly Ca²⁺-dependent. In animal models of stroke, maxipost significantly reduced infarct volume when administered 2 hours after middle cerebral artery occlusion. Thus it may be effective in patients with stroke and neurodegeneration, exerting neuroprotective effects. Others have described maxipost to be a potassium channel opener useful for the treatment or urinary incontinence (WO 02/032419), which is herein incorporated by reference.

### 4.1.4 Further Modulators

Other neuronal potassium channel activators are also useful for the prevention and treatment of dystonia as described above.

This relates especially to activators of KCNQ channels with focus on the subunits 2 to 5 channels and heteromultimers thereof, activators of calcium-activated potassium channels (large conductance and small conductance calcium-activated potassium channels) and activators of G-protein coupled inwardly rectifying potassium channels.

KCNQ channel activators activating KCNQ 2 to KCNQ5 channels are described in WO 2002/000217, WO 2002/066036, WO 2002/066426, WO 2002/072088, WO 2002/096858, WO 2004/047739, WO 2004/047745, WO 2004/047744, WO 2004/047743, WO 2004/047738, WO 2004/058739, WO 2004/058704, WO 2004/060281, WO 2004/060880, WO 2004/080950, WO 2004/080377, WO 2005/025293, WO 2005/087754 which are herein incorporated by reference.

Calcium-activated potassium channels: One large family is the Ca²⁺-activated K⁺ channels, which play important functions in neuronal activity and transepithelial transport. This ion channel family is divided into three groups based on the channels' single channel conductance. These are BK channels (Big-conductance K⁺ channels), IK channels (Intermediate-conductance K⁺ channels), and SK channels (Small-conductance K⁺ channels). While BK channels and SK channels are present in the CNS, IK channels are not present in the CNS.

BK channel activators are described in EP 0747354, WO 1998/016222, WO 2002/030868, EP 0 477 819, EP 0 617 023 and WO 1999/036068. Other compounds including A-411873 are described by Gopalakrishnan and Shieh (Gopalakrishnan M, Shieh C C. Potassium channel subtypes as molecular targets for overactive bladder and other urological disorders. Expert Opin Ther Targets. 2004 October; 8(5):437-58.) These documents are herein incorporated by reference.

The SK channels underlie the medium-duration after-hyperpolarization (mAHP) that follows action potentials in neurons and other excitable cell types. The mAHP functions to set the interspike interval in tonically firing neurons, controlling the action potential firing pattern of neurons. Few activators are known to date, but one example compound is NS309 and its follow-on compound NS4591, both from Neurosearch AS, which, however, are also active as IK channel activators (Strøbaek et al. (2004) Biochim. Biophys. Acta 1665: 1-5), which is herein incorporated by reference.

Other G-protein coupled neuronal potassium channel activators of A-type potassium channels which are activated for example by KW7158 and its analogues are described in WO 1998/046587, which is herein incorporated by reference.

### 4.2. Chemical Forms

The present invention is not limited to any particular chemical form of flupirtine, retigabine or maxipost and the drug may be given to patients either as a free base or as a pharmaceutically acceptable acid addition salt. In the latter case, the hydrochloride salt is generally preferred but other salts derived from organic or inorganic acids may be also used. Examples of such acids include, without limitation, hydrobromic acid, phosphoric acid, sulphuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, and the like. The preparation of flupirtine, 2-amino-3-carbethoxyamino-6-(4-fluorobenzylamino)-pyridine, and its physiologically acceptable salts is described in German patents 1,795,858 and 3,133,519. The preparation of retigabine (2-amino-4-(4-fluorobenzylamino) 1-1-ethoxycarbonylaminobenzene, also designated as N-(2-amino-4-(4-fluorobenzylamino)-phenyl)-carbamic acid ethyl ester) is described in U.S. Pat. No. 5,384,330. The preparation of Maxipost (BMS 204352, 3-(5-chloro-2-methoxyphenyl)-1,3-dihydro-3-fluoro-6-(trifluoromethyl)-2H-indole-2-one) is described in WO 98/16222. The present invention extends also to currently unknown activators of neuronal potassium channels, especially for the family of inward rectifying potassium channels which are activated by flupirtine, retigabine or Maxipost.

### 4.3. Dosage

The total daily dosage of flupirtine, retigabine or maxipost administered to a patient should be at least the amount required to prevent, reduce or eliminate one or more of the symptoms associated with dystonia. The typical daily dosage will be between 20 and 400 mg and, in general, the daily dosage should not exceed 1600 mg. Higher doses are tolerated by some patients and daily dosages of 2,000 mg or more may be considered in refractory cases or in patients receiving concomitant drug treatment with agents may lower the serum concentration and half-life of flupirtine, retigabine or maxipost (e.g., cytochrome P450 inducing compounds such as carbamacepine, phenyloin, phenobarbital and rifampin) as well as in cigarette smokers. In contrast, elderly patients, patients with renal or hepatic dysfunction, and patients receiving concomitant drugs which inhibit the cytochrome P450 system should receive lower initial and maintenance doses, e.g., 5 to 200 mg. These dosage are simply guidelines and the actual dose selected for an individual patient will be determined by the attending physician based upon clinical conditions and using methods well-known in the art. Flupirtine, retigabine or maxipost may be provided in either a single or multiple dosage regimen or on an as needed regime. Examples are: a patient may take 100 mg of flupirtine, retigabine or maxipost orally three times a day or alternatively 200 mg of flupirtine, retigabine or maxipost twice a day. A once daily administration may also be possible, based on the individual symptoms and the extent and duration of relief achieved. A controlled release formulation as described in EP 0615754 for flupirtine as an example, a cutaneous form as described in DE 10255415 A1 for flupirtine as an example or other formulations may as well be used, but a clinical effect in the said diseases is not dependent on the use of these specific dosage forms.

### 4.4. Dosage Forms and Route of Administration

Any route of administration and dosage form is compatible with the present invention and flupirtine, retigabine or maxipost may be administered as either the sole active agent or in combination with other therapeutically active drugs used to treat symptoms of dystonia or to reduce the progression of dystonia. Although compositions suitable for oral delivery are preferred, other routes that may be used include peroral, internal, pulmonary, rectal, nasal, vaginal, lingual, transdermal, intravenous, intraarterial, intramuscular, intraperitoneal, intracutaneous and subcutaneous routes. Specific dosage forms include tablets, pills, capsules, powders, aerosols, suppositories, skin patches, parenterals, and oral liquids including oil aqueous suspensions, solutions and emulsions. Sustained release dosage forms may be used. All dosage forms may be prepared using methods that are standard in the art (see e.g., Remington's Pharmaceutical Sciences, 16th ed., A. Oslo Editor, Easton Pa. (1980)). Specific guidance for the preparation of dosage forms for various routes of delivery is provided by U.S. Pat. Nos. 4,668,684; 5,503,845; and 5,284,861.

The neuronal potassium channel activators including flupirtine, retigabine or maxipost may be used in conjunction with any of the vehicles and excipients commonly employed in pharmaceutical preparations, e.g., talc, gum arabic, lactose, starch, magnesium stearate cocoa butter, aqueous or non-aqueous solvents, oils, paraffin derivates, glycols, ets. Coloring and flavouring agents may also be added to preparations, particularly to those for oral administration. Solution can be prepared using water or physiological compatible organic solvents such as ethanol, 1,2-propylene glycol, polyglycols, dimenthyl sulfoxide, fatty alcohols, triglycerides, partial esters of glycerine and the like. Parenteral compositions containing flupirtine, retigabine or maxipost may be prepared using conventional techniques and include sterile isotonic saline, water, 1,3-butanetiol, ethanol, 1,2-propylene glycol, polyglycols mixed with water, Ringer's solution, etc.

The methods of this invention are useful for inducing, assisting or maintaining desirable treatment effects for patients suffering from dystonia or dystonic symptoms. The method of this invention may be also useful for the prevention of development of dystonic symptoms, either as a result of a developing disease or as a result of drug treatment, for example in patients with Parkinson's disease, psychosis, Huntingdons's disease or Alzheimer's disease. The method of this invention is not limited to the use in human, but may be also used in animals suffering from symptoms of dystonia or a related movement disorder.

The administration of the neuronal potassium channel activator may be as monotherapy or as combination therapy. The neuronal potassium channel activators may be administered in combination with medications registered for the treatment of the underlying disease, i.e. for example Parkinson's disease in the case of L-Dopa induced dyskinesias, or psychosis in the case of neuroleptics induced dystonia, or in combination with muscle relaxants and other drugs useful for the treatment of symptoms of primary or secondary dystonia as listed in the section 2.4, treatment of dystonia.

Example 1: Effect of retigabine and flupiritine in a model of dystonia (study report).

Example 2: Effect of potassium channel openers and blockers in a model of dystonia.

Example 3: Effect of Flupirtine and retigabine as examples for neuronal potassium channel activators in a chronic model of L-DOPA induced dyskinesia

### REFERENCES

Bhatia, K. P., Marsden, C. D., 1994. The behavioural and motor consequences of focal lesions of the basal ganglia in man. Brain 117, 859-876.
Bernardi, H., Lazdunski, M., 1993. Characterization of the sulfonylurea-sensitive ATP-modulated potassium channel. In: H. Glossmann and J. Striessnig, Molecular and cellular biology of pharmacological targets. Plenum Press, New York.
Colatsky, T. J., Follmer, C. H., Starmer, C. F., 1990. Channel specificity in antiarrhythmic drug action. Mechanism of potassium channel block and its role in suppressing and aggravating cardiac arrhythmiasis. Circulation 82, 2235-2242.
Doupnik, C. A., Davidson, N., Lester, H. A., 1995. The inward rectifier potassium channel family. Curr. opin. Neurobiol. 5, 268-277.
Edwards, G., Weston, A. H., 1993. The pharmacology of ATP-sensitive potassium channels. Annual Rev. Pharmacol. Toxicol. 33, 597-637.
Fahn, S., 1995. Medical treatment of dystonia. In: Tsui, J. K. C., Calne, C. B. (Eds.), Handbook of dystonia. Marcel Dekker, New York, pp. 317-328.
Fahn, S., Bressman, S. B., Marsden, C. D., 1998. Classification of dystonia. Adv Neurol 78:1-10.
Greene, P., Kang, U. J., Fahn, S., 1995. Spread of symptoms in idiopathic torsion dystonia. Mov. Disord. 10, 143-152.
Hille, B., 1993, Ionic channels of excitable membranes, 2nd. ed. Sinauer, Sunderland, Mass.
Jankovic, J., 2004. Dystonia: Medical therapy and botulinum toxin. Adv. Neurol. 94, 275-286.
Jankovic, J., Fahn, S., 1998. Dystonic Disorders. In: Jankovic, J., Tolosa, E. (eds.) Parkinson's disease and movement disorders. 3rd Edition. Lipincoft: Philadelphia. 513-551.
Jakob R and Kriegistein J, Influence of flupirtine on a G-protein coupled inwardly rectifying potassium current in hippocampal neurones. Br. J. Pharmacol. 1997, 122:1333-1338.
Kornhuber J., Bleich S, Wiltfang J, Maler M, Parsons C G, Flupirtine shows functional NMDA receptor antagonism by enhancing Mg2+ block via activation of voltage independent potassium channels. Rapid communication. J. Neural Transm. 1999; 106:857-67)
Kubo, Y, Baldwin, T. J., Jan, Y. N., Jan, L. Y., 1993. Primary structure and functional expression of a mouse inward rectifier potassium channel. Nature 362, 127-133.
McKay, M. C., Dworetzky, S. I., Meanwell, N. A., Olesen, S. P., Reinhart, R. H., Levitan, I. B., Adelman, J. P., Gribkoff, V. K., 1994. Opening of large-conductance Calcium-activated potassium channels by the substituted benzimidazolone NS004. J. Neurophysiol. 71, 1873-1882.
Nardocci, N., Fernandez-Alvarez, E., Wood, N. W., Spacy, S. D., Richter, A., 2002. The paroxysmal dyskinesias. In: Guerrini, R., Aicardi, J., Andermann, F., Hallett, M. (Eds.) Epilepsy and Movement Disorders. Cambridge University Press: Cambridge, pp. 125-139.
Nielsen, A. N., Mathiesen, C., Blackburn-Munro, G. (2004) Pharmacological characterisation of acid-induced muscle allodynia in rats. Eur. J. Pharmacol. 487, 93-103.
Richter, A., Löscher, W., 1998. Pathophysiology of idiopathic dystonia: findings from genetic animal models. Progr. Neurobiol. 54, 633-677.
Richter, A., 2005. The genetically dystonic hamster: An animal model of paroxysmal dystonia. In: LeDoux, M. (Ed.) Animal Models of Movement Disorders. Elsevier Academic Press: San Diego, pp. 459-466.
Rudy, B., 1988. Diversity and ubiquity of potassium channels. Neuroscience 25, 729-749.
Rundfeldt C, Netzer R (2000) The novel anticonvulsant retigabine activates M-currents in Chinese hamster ovary-cells transfected with human KCNQ2/3 subunits. Neurosci Lett 282:73-76.
Rupersberg, J. P., Ermler, M., Knopf, M., Kues, W., Jonas, P., Koenen, M., 1993. Properties of shaker-homologues potassium channels expressed in the mammalian brain. Cell Physiol. Biochem. 3, 250-258.
Saunders-Pullman, R., Bressman, S., 2005. Clinical features and classification of the human dystonias. In: LeDoux M (Ed.) Animal Models of Movement Disorders. Elsevier Academic Press: San Diego, pp. 227-240.
Takumi, T., Ohkubo, H., Nakanishi, S., 1988. Cloning of a membrane protein that induces a slow voltage-gated potassium current. Science 242, 1042-1045.
Vennekamp, J., Wulff, H., Beeton, C., Calabresi, P. A., Grissmer, S., Hansel, W., Chandy, K. G., 2004. Kv1.3-Blocking 5-Phenylalkoxypsoralens: A New Class of Immunomodulators. Mol. Pharmacol. 65, 1364-1374.
Vitek J. L., Giroux, M., 2000. Physiology of hypokinetic and hyperkinetic movement disorders: Model for dyskinesia. Ann. Neurol. 47, 131-140.
Wichmann, T., DeLong, M. R., 1996. Functional and pathophysiological models of the basal ganglia. Curr. Opin. Neurobiol. 6, 751-758.

### EXAMPLE 1

### Examinations of Retigabine and Flupirtine in the dt^{sz} Mutant Hamster

Aim: We investigated the anti-dystonic effect of neuronal potassium channel activators in a predictive model of paroxysmal dystonia. To evaluate the role of different neuronal potassium channels, the selective activator of Kv7 channels, retigabine was used. In addition, flupirtine, which is known to activate inward rectifying potassium channels and Kv7 potassium channels, was used.

### Materials and Methods

Animals The dt^{sz} mutant hamsters (Syrian golden hamsters), used in the present experiments, were obtained by selective breeding as described in detail elsewhere (Richter and Löscher, 1998). In this inbred line of mutant hamsters the motor disturbances are transmitted by a recessive gene. All animal groups consisted of male and female hamsters, because there was no indication of sex-related differences in the severity of dystonia or in the response to drugs (Richter and Löscher, 1998). The animals were born and kept under controlled environmental conditions (23-25° C., 50-60% humidity, 13 h light/11 h dark cycle) with free access to standard Altromin 7204 diet and water. In cases of oral administrations the food was deprived for 2 h prior to the experiments. All experiments were carried out in the morning (08.30-12.00 a.m.) at controlled temperatures (23-25° C.).

Induction of dystonic episodes and severity-score of dystonia. As reported previously in detail (for reviews see Richter and Löscher, 1998, Richter, 2005), the dt^{sz} mutant hamster exhibits long-lasting dystonic episodes which can be provoked by mild stress, such as handling. For drug testing, dystonic attacks can be reproducibly induced by a triple stimulation technique (Richter and Löscher, 1998), i.e., stressful stimuli consisting of (1) taking the animal from its home cage and placing it on a balance, (2) intraperitoneal (i.p.) injection of isotonic saline (or of retigabine or flupirtine, see below) or oral administration of isotonic saline via pharyngeal cannulation (or of retigabine, see below), and (3) placement of the animal in a new plastic cage. After this procedure, dt^{sz} hamsters develop a sequence of abnormal movements and postures. Dystonia is the predominant symptom in these animals. The severity of dystonia can be rated by following score-system (Richter and Löscher, 1998): stage 1, flat body posture; stage 2, facial contortions, rearing with forelimbs crossing, disturbed gait with hyperextended forepaws; stage 3, hyperextended hindlimbs so that the animals appear to walk on tiptoes; stage 4, twisting movements and loss of balance; stage 5, hindlimbs hyperextended caudally; stage 6, immobilisation in a twisted, hunched posture with hind- and forelimbs tonically extended forward. After reaching the individual maximum stage the hamsters recover within 2-5 hours. The individual maximum stage of dystonia is usually reached within 3 hours after the hamsters were placed into the new cage. Therefore, the animals have to be observed for 3 h after the induction of dystonic attacks to determine the individual maximum stage reached after administrations of drugs or of vehicle (for pre- and post-drug control recordings).

In the present study, all animals were examined for the presence of dystonia after weaning at the age of 21 days by the triple stimulation procedure, including injections of saline. Dystonia shows an age-dependent time-course with a maximum of the severity of dystonia at an age of 30-42 days (Richter and Löscher, 1998). All groups of mutant hamsters used for investigations were repeatedly tested by triple stimulations (injections of saline) every 2 to 3 days after weaning until the severity of dystonia and latencies to the different stages were reproducible. The drug experiments were done in 30-42 days old hamsters, i.e. at an age of the maximum severity of dystonia.

Drug experiments The effects of the Kᵥ7.2/7.3 channel activators retigabine (5, 7.5 and 10 mg/kg i.p. and 10 and 20 mg/kg p.o.) and flupirtine (10 and 20 mg/kg i.p.) were examined in groups of 6-10 dt^{s/z} hamsters. The total number of animals used for the present experiments was 68. The compounds were freshly dissolved in saline prior the experiments. Dystonic attacks were induced by the procedure of triple stimulation, as described above, but instead of saline the active compound was injected (injection volume: 5 ml/kg i.p. or p.o.). Pre- and post-drug control trials with the vehicle (injection volume: 5 ml/kg saline i.p. or p.o.) were undertaken 2-3 days before and 2-3 days after drug testing in the same animals. Since the individual maximum stage of dystonia is usually reached within 3 h, the hamsters were observed for 3 h after triple stimulation. During this period the severity of dystonia, the latencies to the different stages and the side effects were noted. The rater of the severity score (and of the latencies to stages) was unaware whether the animals were treated with vehicle or an active principle. A second person who had done the preparation of the solutions observed the animals for behavioural effects. The side effects were not quantified, but locomotor activity and ataxia were determined according to a score system, as previously described (Löscher and Richter, 1994). Animals which differed in their individual maximum stage by more than 2 stages between pre-drug and post-drug controls were omitted from evaluation (8 out of 68 animals). In addition, one animal had to be euthanized because of a bad general condition after i.p. injection of retigabine at a high dose of 10 mg/kg.

Significant differences in severity of dystonia and in the latencies to onset of dystonia (latency to stage 2; see Table 1) between control trials (pre- and post-drug) and drug trial in the same group of animals were calculated by the Friedman test and, if there was a significant difference (at least P<0.05) the Wilcoxon signed rank test for paired replicates was used post hoc to determine which pairs differ.

### Results

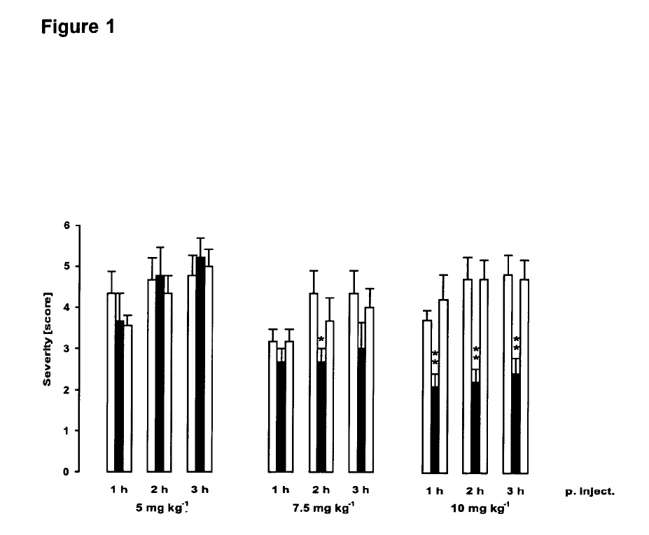

The means ±S.E. of the severity of dystonia after treatment with the KCNQ channel openers (retigabine and flupirtine) are illustrated in FIG. 1-3. The means ±S.E. of the latency to onset of dystonia are summarized in Table 1. Individual data, observed in the experiments with retigabine and flupirtine are shown in Table 2-8. The effects on the severity of dystonia are summarized in Table 2-8 A and the effects on the latency to onset of dystonia are summarized in Table 2-8 B.

As shown in FIG. 1, retigabine exerted a dose-dependent improvement of dystonia after intraperitoneal injections. At a dose of 10 mg/kg, retigabine significantly suppressed the progression of dystonia (see first and second h after administration) and significantly reduced the maximum severity (see third h after injection), while a lower dose of 7.5 mg/kg only tended to reduce the severity as indicated by a significant decrease of the severity which was restricted to the second h after injection. At a dose of 5 mg/kg, retigabine failed to exert any significant effects on the severity of dystonia. A complete prevention was observed in one hamster treated with 10 mg/kg (see Table 4A). Retigabine increased the latency to onset of dystonia at a dose of 7.5 mg/kg (Table 1), while 5 and 10 mg/kg merely tended to delay the onset of dystonic episodes (see also Table 2-4B). Behavioural effects were a moderate to unequivocal hypolocomotion (sometimes interrupted by short lasting periods of increased locomotor activity) and ataxia within the first h after administration. The hamsters writhed with pain during the first 5 min after injection of 10 mg/kg. Four hamsters which received 5 mg/kg i.p. five days after treatment with 10 mg/kg i.p. showed a bad general condition. While three of these hamsters recovered within 2 to 3 days, one animal had to be euthanized. Obduction indicated a dilated colon.

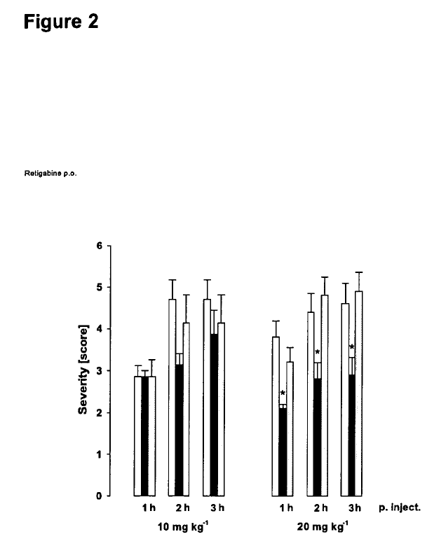

The abdominal adverse effects after i.p. injections of retigabine prompted us to examine the effects of retigabine after oral administration. As shown in FIG. 2, retigabine significantly reduced the severity of dystonia at an oral dose of 20 mg/kg, while oral administration of 10 mg/kg failed to exert antidystonic effects. At both oral doses, retigabine did not exert significant effects on the latency to onset of dystonia (Table 1). In contrast to the observations after intraperitoneal injections, retigabine did not exert severe side effects at the oral doses of 10 and 20 mg/kg. Two hamsters treated with 10 mg/kg p.o. showed a moderate reduction of the locomotor activity. At a higher dose of 20 mg/kg p.o., seven animals exhibited moderate ataxia and five hamsters showed moderate hypolocomotion during the first hour after administration.

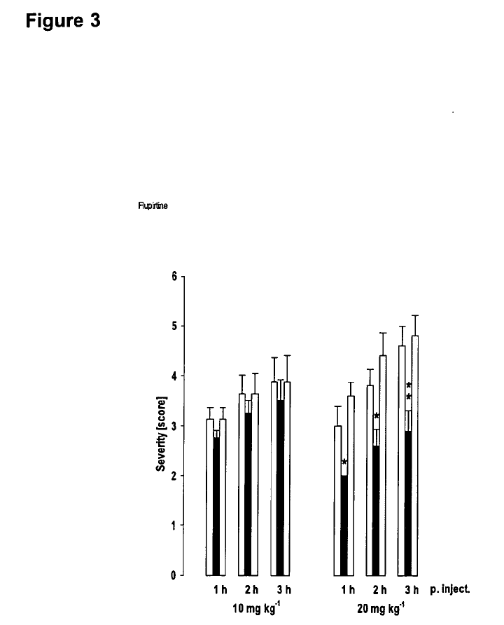

As shown in FIG. 3, flupirtine did not exerted significant antidystonic effects a dose of 10 mg/kg i.p. At a higher dose of 20 mg/kg i.p., flupirtine delayed the progression of dystonia (first and second h), reduced the maximum severity (third h) and increased the latency to onset of dystonia, indicating a fast onset of action (FIG. 3, Table 1). Adverse effects were a moderate hypolocomotion and ataxia within the first hour after administration of 10 mg/kg. At a dose of 20 mg/kg, flupirtine caused a more marked ataxia (lasting up to 90 min) and an unequivocal hypolocomotion (5 to 15 min after injection) followed by hyperlocomotion (15-60 min after injection).

### Conclusions

The present data demonstrate for the first time beneficial effects of the neuronal potassium channel activators retigabine and flupirine in an animal model of paroxysmal dyskinesia. These data suggest that dysfunctions of neuronal potassium channels including Kᵥ7.2/7.3 channels, G-protein coupled inward-rectifying potassium channels and other neuronal potassium channels including Bkmax deserve attention in the research of the pathophysiology of dyskinesias. Antidystonic efficacy was found at well tolerated doses of retigabine (at least after oral administration) and of flupirtine. Since the antiepileptic drug retigabine as well as the analgetic flupirtine are well tolerated in humans (Fatope, 2001), the present finding of pronounced antidystonic efficacy of these neuronal potassium channel activators in the hamster model suggests that respective compounds including retigabine and flupirtine may provide novel therapeutic approaches for paroxysmal dyskinesias. Furthermore, the well-known efficacy of Kᵥ7.2/7.3 channel openers and flupirtine against neuropathic or muscle-mediated pain might contribute to improvement of this disorder because the dystonic syndrome is often accompanied by painful muscle spasms (Nielsen et al., 2004).

In contrast to paroxysmal dystonia, other types of paroxysmal dyskinesias (nocturnal dyskinesias, paroxysmal kinesiogenic dyskinesias) can coexist with epilepsy in the same individual or family (Du et al., 2005; Guerrini, 2001). In view to the well known anticonvulsant effects of retigabine and the here demonstrated antidystonic activity, Kᵥ7.2/3 channel activators may be also interesting candidates for the treatment of other types of hereditary dyskinesias.

Kᵥ7 (7.2, 7.3 and 7.5) channels are expressed in medium spiny neurons. These channels have been reported to be potent regulators of the excitability of these projection neurons (Shen et al., 2005). They are modulated by striatal cholinergic interneurons, i.e., increased cholinergic tone can result in a reduction of Kᵥ7 channel opening in medium spiny neurons, increasing their excitability. Changes in Kᵥ7 (Kᵥ7.2, 7.3 or 7.5) channels or of other neuronal potassium channels may be therefore important in basal ganglia diseases. Despite a different primary defect in various types of dystonias and dyskinesias, there are possibly common mechanisms leading to dystonic disturbances. There is evidence that the dystonic syndrome in patients with primary dystonia as well as in hereditary dyskinesias is associated with increased striatal activity leading to reduced basal ganglia output (Bennay et al., 2001, Gernert et al., 2002; Vitek, 2002; Yamada et al., 2005). Therefore, neuronal potassium channel activators and especially Kᵥ7 channel openers including but not limited to retigabine, flupirtine and maxipost, may be effective in various types of dystonias and dyskinesias, including levodopa-induced dyskinesias.

It has to be noted, that retigabine has been found to potentiate the action of γ-aminobutyric acid (GABA) and to reduce brain levels of glutamate (Rundfeldt and Netzer, 2000b; Sills et al., 2000). In view of antidystonic effects of GABA-potentiating drugs in the dt^{sz} hamster, the effects of Kᵥ7 channel blockers alone and together with retigabine have to clarify if its antidystonic effect is mainly mediated by the opening of Kᵥ7 channels. These experiments will be done independently on the cooperation contract.

### REFERENCES

Bennay M, Gernert M, Richter A (2001) Spontaneous remission of paroxysmal dystonia coincides with normalization of entopeduncular activity in dt^{sz} mutants. J Neurosci 21, RC153 (14).
Du W, Bautista J F, Yang H, Diez-Sampedro A, You S A, Wang L, Kotagal P, Luders H O, Shi J, Cui J, Richerson G B, Wang Q K (2005) Calcium-sensitive potassium channelopathy in human epilepsy and paroxysmal movement disorder. Nat Genet. 37, 733-738.
Fatope M O (2001) Retigabine (ASTA Medica). Drugs 4, 93-98.
Gernert M, Hamann M, Bennay M, Löscher W, Richter A (2000) Deficit of striatal parvalbumin-reactive GABAergic interneurons and decreased basal ganglia output in a genetic rodent model of idiopathic paroxysmal dystonia. J Neurosci 20, 7052-7058.
Guerrini R (2001) Idiopathic epilepsy and paroxysmal dyskinesia. Epilepsia. 42 Suppl 3, 36-41.
Jankovic J, Demirkiran M (2002) Classification of paroxysmal dyskinesias and ataxias. In: Fahn S, Frucht S J, Hallett M, Truong, D D (Eds) Myoclonus and paroxysmal dyskinesia. Adv Neurol 89, 387-400.
Korsgaard M P, Hartz B P, Brown W D, Ahring P K, Strobaek D, Mirza N R (2005) Anxiolytic effects of Maxipost (BMS-204352) and retigabine via activation of neuronal Kᵥ7 channels. J Pharmacol Exp Ther 314, 282-292.
Lawson K (2000) Is there a role for potassium channel openers in neuronal ion channel disorders? Expert Opin Investig Drugs 9, 2269-2280.
Löscher W, Richter A (1994) The novel selective and silent 5-HT_{1A} receptor antagonist (+)-WAY-100135 aggravates dystonic movements in a mutant hamster model. Eur J Pharmacol 255, 235-238.
Nardocci N, Fernandez-Alvarez E, Wood N R, Spacy S D, Richter A (2002) The paroxysmal dyskinesias. In: Guerrini R, Aicardi J, Andermann F, Hallett M (Eds.) Epilepsy and Movement Disorders. Cambridge University Press: Cambridge, S. 125-139.
Nielsen A N, Mathiesen C, Blackburn-Munro G (2004) Pharmacological characterisation of acid-induced muscle allodynia in rats. Eur J Pharmacol 487, 93-103.
Ptacek L J (1997) Channelopathies: ion channel disorders of muscle as a paradigm for paroxysmal disorders of the nervous system. Neuromuscul Disord 7, 250-255.
Richter, A, Löscher, W (1998) Pathophysiology of idiopathic dystonia: findings from genetic animal models. Prog. Neurobiol. 54, 633-677.
Richter A, Löscher W (2002) Animal models of paroxysmal dyskinesia. Adv Neurol 89, 443-451.
Richter A (2005) The genetically dystonic hamster: An animal model of paroxysmal dystonia. In: LeDoux M (Ed.) Animal Models of Movement Disorders. Elsevier Academic Press: San Diego, pp. 459-466.
Rundfeldt C, Netzer R (2000a) The novel anticonvulsant retigabine activates M-currents in Chinese hamster ovary-cells transfected with human KCNQ2/3 subunits. Neurosci Lett 282, 73-76.
Rundfeldt C, Netzer R (2000b) Investigations into the mechanism of action of the new anticonvulsant retigabine. Interaction with GABAergic and glutamatergic neurotransmission and with voltage gated ion channels. Drug Res (Arzneimittelforschung) 50, 1063-70.
Shen W, Hamilton S E, Nathanson N M, Surmeier D J (2005) Cholinergic suppression of KCNQ channel currents enhances excitability of striatal medium spiny neurons. J Neurosci 25, 7449-7458.
Shieh C C, Coghlan M, Sullivan J P, Gopalakrishnan M (2000) Potassium channels: molecular defects, diseases, and therapeutic opportunities. Pharmacol Rev. 52, 557-94.
Sills G J, Rundfeldt C, Butler E, Forrest G, Thompson G G, Brodie M J (2000) A neurochemical study of the novel antiepileptic drug retigabine in mouse brain. Pharmacol Res. 42, 553-557.
Vitek, J L (2002) Pathophysiology of dystonia: a neuronal model. Movement Dis 17 (Suppl. 3), S49-S62.
Yamada K, Goto S, Soyama N, Shimoda O, Kudo M, Kuratsu J I, Murase N, Kaji R. (2005) Complete suppression of paroxysmal nonkinesigenic dyskinesia by globus pallidus internus pallidal stimulation. Mov Disord, Epub

TABLE 1
Effects of the neuronal potassium channel openers retigabine
and flupirtine on the latency to onset of dystonia. Latency on
was determined as the time to the first unequivocal signs of
the dystonic attacks (stage 2).

| Dose [mg/kg]/ | | Latency to onset (min) | | | |
|---|---|---|---|---|---|
| (Route of administration) | | Pre-Drug | Drug | Post-Drug | (n) |
| Retigabine | | | | | |
| 5.0 | (i.p.) | 6.9 ± 2.4 | 27.1 ± 17.7 | 4.0 ± 1.1 | 9 |
| 7.5 | (i.p.) | 6.0 ± 1.1 | 11.8 ± 1.0* | 5.0 ± 1.2 | 6 |
| 10.0 | (i.p.) | 3.7 ± 0.6 | 7.1 ± 2.9 | 4.4 ± 1.2 | 9 |
| 10.0 | (p.o) | 2.0 ± 0.6 | 4.9 ± 2.9 | 5.1 ± 1.9 | 7 |
| 20.0 | (p.o.) | 4.0 ± 0.6 | 9.6 ± 2.0 | 3.1 ± 0.7 | 10 |
| Flupirtine | | | | | |
| 10.0 | (i.p.) | 6.0 ± 0.7 | 6.9 ± 1.6 | 4.6 ± 0.9 | 8 |
| 20.0 | (i.p.) | 1.8 ± 0.3 | 13.8 ± 3.8* | 4.1 ± 0.8 | 10 |

Data are shown as means ± S.E. of the number of animals indicated (n).
Significant differences to pre-drug and post-drug controls are marked by asterisk (*P < 0.05).

FIG. 1. Effect of retigabine on severity of dystonia in mutant hamsters after intraperitoneal (i.p.) injections of 5.0, 7.5 and 10 mg/kg. The white bars in each set of three bars indicate the control values obtained two days before drug administration (pre drug control). The black bar refers to the day of drug administration in the same animal groups. The gray bars in each set of three bars indicate the control values obtained two days after drug administration (post drug control). The individual maximum severity of dystonia is usually reached within 3 h after induction of dystonia by triple stimulation including the injection of drugs or vehicle. The figure shows the average of the maximum individual severity scores of dystonia reached within the 1st, 2nd and 3rd h post injection of isotonic saline (control trials) or of retigabine, reflecting the progression of dystonia in dt^{sz} hamsters during control recordings and after treatment with the active compound. Asterisks indicate significant reduction of dystonia in comparison to the pre- and post-drug control (*P<0.05, **P<0.01; P-values of one-sided tests). Data are shown as means ±S.E. (number of animals: see Table 1).

FIG. 2. Effect of retigabine on severity of dystonia in mutant hamsters after oral administration of 10 and 20 mg/kg. The white bars in each set of three bars indicate the control values obtained two days before drug administration (pre drug control). The black bar refers to the day of drug administration in the same animal groups. The gray bars in each set of three bars indicate the control values obtained two days after drug administration (post drug control). The figure shows the average of the maximum individual severity scores of dystonia reached within the 1st, 2nd and 3rd h after oral administration via a pharyngeal cannulation of isotonic saline (control trials) or of retigabine. Asterisks indicate significant reduction of dystonia in comparison to the pre- and post-drug control (*P<0.01; P-values of one-sided tests). Data are shown as means ±S.E. (number of animals: see Table 1). For further explanations see FIG. 1 legend.

FIG. 3: Effect of flupirtine on severity of dystonia in mutant hamsters after intraperitoneal (i.p.) injections of 10 and 20 mg/kg. The white bars in each set of three bars indicate the control values obtained two days before drug administration (pre drug control). The black bar refers to the day of drug administration in the same animal groups. The gray bars in each set of three bars indicate the control values obtained two days after drug administration (post drug control). The figure shows the average of the maximum individual severity scores of dystonia reached within the 1st, 2nd and 3rd h post injection of isotonic saline (control trials) or of retigabine. Asterisks indicate significant reduction of dystonia in comparison to the pre- and post-drug control (*P<0.05, **P<0.01; P-values of one-sided tests). Data are shown as means ±S.E. (number of animals: see Table 1).

### Legend to Tables 2-8:

Individual data are shown in Table 2-8.
A. Maximum individual severity scores of dystonia reached within the 1st, 2nd and 3rd h after administration of the Kᵥ7 channel openers (drug) or of the vehicle for pre- and post-drug control recording.
B. Latencies to onset of dystonic attacks (Latency on) and latencies to stage 6 (Latency max) in genetically dystonic hamsters after treatment with the Kᵥ7 channel openers (drug) or with vehicle for pre- and post-drug control recording. Latency on was determined as the time (min) to the first unequivocal signs of dystonic attacks (stage 2). Data on the latency to onset provide information about the onset of action.

TABLE 2
Retigabine 5 mg/kg (soluted in 0.9% NaCl) i.p.; age: 37-39 days of
life
2A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 159/2 | 6 | 5 | 4 | 6 | 5 | 4 | 6 | 6 | 6 |
| dt 159/3 | 6 | 6 | 4 | 6 | 6 | 6 | 6 | 6 | 6 |
| dt 159/4 | 6 | 3 | 4 | 6 | 6 | 4 | 6 | 6 | 6 |
| dt 164/3 | 2 | 3 | 3 | 3 | 5 | 3 | 3 | 5 | 4 |
| dt 164/6 | 3 | 3 | 3 | 3 | 3 | 3 | 4 | 4 | 4 |
| dt 169/5 | 6 | 5 | 3 | 6 | 6 | 6 | 6 | 6 | 6 |
| dt 169/7 | 3 | 2 | 3 | 3 | 6 | 4 | 3 | 6 | 4 |
| dt 169/9 | 4 | 6 | 5 | 6 | 6 | 6 | 6 | 6 | 6 |
| dt 169/11 | 3 | 0 | 3 | 3 | 0 | 3 | 3 | 2 | 3 |
| Mean | 4.3 | 3.7 | 3.6 | 4.7 | 4.8 | 4.3 | 4.8 | 5.2 | 5.0 |
| S.E. | 0.6 | 0.7 | 0.2 | 0.5 | 0.7 | 0.4 | 0.5 | 0.5 | 0.4 |
| S.D. | 1.7 | 2.0 | 0.7 | 1.6 | 2.1 | 1.3 | 1.5 | 1.4 | 1.2 |

| | |
|---|---|
| Friedman (P=): | 1^{st} h 0.569, 2^{nd} h 0.685, 3^{rd} h 0.814 (not significant) |
| Wilcoxon (P=): | one-sided/two-sided: (one-sided: not significant, |
| | two-sided: not significant) |
| | vs. pre-drug: 1^{st} h 0.149/0.297, 2^{nd} h 0.438/0.875, |
| | 3^{rd} h 0.250/0.5 |
| | vs. post-drug: 1^{st} h 0.407/0.813, 2^{nd} h 0.313/0.625, |
| | 3^{rd} h 0.250/0.5 |

2B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt 159/2 | 2 | 1 | 2 | 43 | — | 133 |
| dt 159/3 | 1 | 1 | 1 | 30 | 43 | 61 |
| dt 159/4 | 3 | 1 | 1 | 46 | — | 132 |
| dt 164/3 | 24 | 23 | 7 | — | — | — |
| dt 164/6 | 6 | 15 | 4 | — | — | — |
| dt 169/5 | 5 | 9 | 9 | 48 | — | — |
| dt 169/7 | 5 | 15 | 8 | — | 87 | — |
| dt 169/9 | 4 | 12 | 1 | 60 | 39 | — |
| dt 169/11 | 12 | 167 | 3 | — | — | — |
| Mean | 6.9 | 27.1 | 4.0 | n.d. | n.d. | n.d. |
| S.E. | 2.4 | 17.7 | 1.1 | n.d. | n.d. | n.d. |
| S.D. | 7.2 | 53.0 | 3.2 | n.d. | n.d. | n.d. |

| | | |
|---|---|---|
| Latency on: | Friedmann: | P = 0.328 (not significant) |
| | Wilcoxon: | vs. pre-drug: one-sided P = 0.055, two-sided |
| | | P = 0.109 |
| | | vs. post-drug: one-sided P = 0.032, |
| | | two-sided P = 0.063 |
| | | (one-sided: not significant vs. pre- and |
| | | post-drug control) |

Latency max: not determined (n.d.) because less than 5 animals reached
stage 6 during drug and control trials

TABLE 3
Retigabine 7.5 mg/kg (soluted in 0.9% NaCl) i.p.; age: 38 days
of life
3A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 171/1 | 3 | 3 | 3 | 4 | 3 | 4 | 4 | 3 | 4 |
| dt 171/2 | 4 | 4 | 4 | 6 | 4 | 6 | 6 | 6 | 6 |
| dt 171/4 | 3 | 2 | 3 | 3 | 2 | 3 | 3 | 2 | 3 |
| dt 171/5 | 3 | 2 | 2 | 3 | 2 | 2 | 3 | 2 | 3 |
| dt 171/6 | 2 | 2 | 3 | 6 | 2 | 3 | 6 | 2 | 4 |
| dt 171/7 | 4 | 3 | 4 | 4 | 3 | 4 | 4 | 3 | 4 |
| Mean | 3.2 | 2.7 | 3.2 | 4.3 | 2.7 | 3.7 | 4.3 | 3.0 | 4.0 |
| S.E. | 0.3 | 0.3 | 0.3 | 0.6 | 0.3 | 0.6 | 0.6 | 0.6 | 0.5 |
| S.D. | 0.8 | 0.8 | 0.8 | 1.4 | 0.8 | 1.4 | 1.4 | 1.6 | 1.1 |

| | |
|---|---|
| Friedman (P=): | 1^{st} h 0.430, 2^{nd} h 0.012 (significant), 3^{rd} h 0.052 |
| Wilcoxon (P=): | one-sided/two-sided: (one-sided: 2^{nd} h significant, 3^{rd} h |
| | not significant because of Friedman, two-sided: 2^{nd} h not |
| | significant vs. pre- and post-drug control) |
| | vs. pre-drug: 1^{st} h 0.125/0.25, 2^{nd} h 0.016/0.031, 3^{rd} h |
| | 0.032/0.063 |
| | vs. post-drug: 1^{st} h 0.125/0.25, 2^{nd} h 0.032/0.063, 3^{rd} h |
| | 0.032/0.063 |

3B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt 171/1 | 5 | 12 | 6 | — | — | — |
| dt 171/2 | 8 | 11 | 3 | 69 | 149 | — |
| dt 171/4 | 10 | 15 | 3 | — | — | — |
| dt 171/5 | 5 | 10 | 6 | — | — | — |
| dt 171/6 | 6 | 9 | 2 | 112 | — | — |
| dt 171/7 | 2 | 14 | 10 | — | — | — |
| Mean | 6.0 | 11.8 | 5.0 | n.d. | n.d. | n.d. |
| S.E. | 1.1 | 1.0 | 1.2 | n.d. | n.d. | n.d. |
| S.D. | 2.8 | 2.3 | 3.0 | n.d. | n.d. | n.d. |

| | | |
|---|---|---|
| Latency on: | Friedmann: | P = 0.008 (significant) |
| | Wilcoxon: | vs. pre-drug: one-sided P = 0.016, two-sided |
| | | P = 0.031 (significant) |
| | | vs. post-drug: one-sided P = 0.016, |
| | | two-sided P = 0.031 (significant) |

Latency max: not determined (n.d.) because less than 5 animals reached
stage 6 during drug and control trials

TABLE 4
Retigabine: 10 mg/kg (soluted in 0.9% NaCl) i.p.; age: 32-39
days of life
4A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 154/4 | 3 | 2 | 3 | 3 | 2 | 3 | 4 | 2 | 3 |
| dt 154/5 | 5 | 2 | 4 | 6 | 2 | 5 | 6 | 3 | 5 |
| dt 154/10 | 3 | 2 | 2 | 3 | 2 | 3 | 3 | 2 | 3 |
| dt 155/1 | 3 | 3 | 3 | 3 | 3 | 4 | 3 | 3 | 4 |
| dt 155/6 | 3 | 0 | 2 | 3 | 0 | 3 | 3 | 0 | 3 |
| dt 159/2 | 4 | 2 | 6 | 6 | 3 | 6 | 6 | 3 | 6 |
| dt 159/3 | 4 | 3 | 6 | 6 | 3 | 6 | 6 | 3 | 6 |
| dt 159/4 | 4 | 2 | 6 | 6 | 2 | 6 | 6 | 2 | 6 |
| dt 159/5 | 4 | 3 | 6 | 6 | 3 | 6 | 6 | 4 | 6 |
| Mean | 3.7 | 2.1 | 4.2 | 4.7 | 2.2 | 4.7 | 4.8 | 2.4 | 4.7 |
| S.E. | 0.2 | 0.3 | 0.6 | 0.5 | 0.3 | 0.5 | 0.5 | 0.4 | 0.5 |
| S.D. | 0.7 | 0.9 | 1.8 | 1.6 | 1.0 | 1.4 | 1.5 | 1.1 | 1.4 |

| | |
|---|---|
| Friedman: | 1^{st} h P = 0.006 (significant), 2^{nd} h P < 0.01(significant), |
| | 3^{rd} h P < 0.001 (significant) |
| Wilcoxon (P=): | one-sided/two-sided: (one-sided: significant, two-sided: |
| | significant) |
| | vs. pre-drug: 1^{st} h 0.004/0.008, 2^{nd} h 0.004/0.008, 3^{rd} h |
| | 0.004/0.008 |
| | vs. post-drug: 1^{st} h 0.008/0.016, 2^{nd} h = 0.002/0.004, 3^{rd} h |
| | 0.002/0.004 |

4B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt 154/4 | 5 | 6 | 4 | — | — | — |
| dt 154/5 | 3 | 14 | 10 | 85 | — | — |
| dt 154/10 | 3 | 25 | 10 | — | — | — |
| dt 155/1 | 1 | 3 | 2 | — | — | — |
| dt 155/6 | 1 | — | 7 | — | — | — |
| dt 159/2 | 5 | 3 | 2 | 92 | — | 43 |
| dt 159/3 | 5 | 3 | 1 | 106 | — | 30 |
| dt 159/4 | 5 | 1 | 3 | 91 | — | 46 |
| dt 159/5 | 5 | 2 | 1 | 70 | — | 41 |
| Mean | 3.7 | 7.1 | 4.4 | n.d. | n.d. | n.d. |
| S.E. | 0.6 | 2.9 | 1.2 | n.d. | n.d. | n.d. |
| S.D. | 1.7 | 8.3 | 3.6 | n.d. | n.d. | n.d. |

| | | |
|---|---|---|
| Latency on: | Friedman: | P = 0.236 (not significant) |
| | Wilcoxon: | vs. pre-drug: one-sided P = 0.473 two-sided |
| | | P = 0.945 |
| | | vs. post-drug: one-sided P = 0.039, |
| | | two-sided P = 0.078 |
| | | (one-sided: not significant vs. pre- and |
| | | post-drug control) |

Latency max: not determined (n.d.) because less than 5 animals reached
stage 6 during drug and control trials

TABLE 5
Retigabine 10 mg/kg (soluted in 0.9% NaCl) p.o.; age: 37-40
days of life
5A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 200/1 | 4 | 3 | 2 | 6 | 3 | 6 | 6 | 6 | 6 |
| dt 200/4 | 2 | 3 | 2 | 4 | 3 | 3 | 4 | 3 | 3 |
| dt 200/6 | 2 | 3 | 3 | 6 | 4 | 6 | 6 | 6 | 6 |
| dt 202/10 | 3 | 3 | 2 | 3 | 3 | 2 | 3 | 3 | 2 |
| dt 204/1 | 3 | 3 | 3 | 4 | 3 | 3 | 4 | 3 | 3 |
| dt 204/3 | 3 | 2 | 3 | 4 | 2 | 3 | 4 | 2 | 3 |
| dt 204/6 | 3 | 3 | 5 | 6 | 4 | 6 | 6 | 4 | 6 |
| Mean | 2.9 | 2.9 | 2.9 | 4.7 | 3.1 | 4.1 | 4.7 | 3.9 | 4.1 |
| S.E. | 0.3 | 0.1 | 0.4 | 0.5 | 0.3 | 0.7 | 0.5 | 0.6 | 0.7 |
| S.D. | 0.7 | 0.4 | 1.1 | 1.3 | 0.7 | 1.8 | 1.3 | 1.6 | 1.8 |

| | |
|---|---|
| Friedman (P=): | 1^{st} h 0.964, 2^{nd} h 0.027 (significant), 3^{rd} h 0.192 |
| Wilcoxon (P=): | one-sided/two-sided: (one-sided: 2^{nd} h not significant |
| | vs. pre- and post-drug control, 1^{st} and 3^{rd} h not |
| | significant, two-sided: not significant) |
| | vs. pre-drug: 1^{st} h 0.5/1.0, 2^{nd} h 0.016/0.031, 3^{rd} h |
| | 0.063/0.125 |
| | vs. post-drug: 1^{st} h 0.5/1.0, 2^{nd} h 0.063/0.125, 3^{rd} h |
| | 0.25/0.5 |

5B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt | 2 | 1 | 3 | 81 | 168 | 73 |
| 200/1 | | | | | | |
| dt | 1 | 22 | 16 | — | — | — |
| 200/4 | | | | | | |
| dt | 1 | 1 | 1 | 111 | 148 | 70 |
| 200/6 | | | | | | |
| dt | 5 | 3 | 3 | — | — | — |
| 202/10 | | | | | | |
| dt | 1 | 2 | 5 | — | — | — |
| 204/1 | | | | | | |
| dt | 1 | 3 | 3 | — | — | — |
| 204/3 | | | | | | |
| dt | 3 | 2 | 5 | 90 | — | 87 |
| 204/6 | | | | | | |
| Mean | 2.0 | 4.9 | 5.1 | n.d. | n.d. | n.d. |
| S.E. | 0.6 | 2.9 | 1.9 | n.d. | n.d. | n.d. |
| S.D. | 1.5 | 7.6 | 5.0 | n.d. | n.d. | n.d. |

| | | |
|---|---|---|
| Latency on: | Friedman: | P = 0.486 (not significant) |
| | Wilcoxon: | vs. pre-drug: one-sided P = 0.344, two-sided |
| | | P = 0.688 |
| | | vs. post-drug: one-sided P = 0.438, |
| | | two-sided P = 0.875 |
| | | (one-sided: not significant) |

Latency max: not determined (n.d.) because less than 5 animals reached
stage 6 during drug and control trials

TABLE 6
Retigabine: 20 mg/kg (soluted in 0.9% NaCl) p.o.; age: 32-36
days of life
6A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 205/2 | 3 | 2 | 3 | 6 | 3 | 6 | 6 | 4 | 6 |
| dt 205/7 | 6 | 2 | 4 | 6 | 6 | 6 | 6 | 6 | 6 |
| dt 205/9 | 6 | 2 | 3 | 6 | 3 | 6 | 6 | 3 | 6 |
| dt 206/2 | 3 | 3 | 3 | 3 | 3 | 3 | 3 | 3 | 3 |
| dt 206/7 | 4 | 2 | 6 | 6 | 2 | 6 | 6 | 2 | 6 |
| dt 206/8 | 3 | 2 | 2 | 4 | 2 | 6 | 6 | 2 | 6 |
| dt 206/9 | 4 | 2 | 3 | 4 | 3 | 5 | 4 | 3 | 6 |
| dt 206/10 | 3 | 2 | 2 | 3 | 2 | 4 | 3 | 2 | 4 |
| dt 207/2 | 3 | 2 | 3 | 3 | 2 | 3 | 3 | 2 | 3 |
| dt 207/13 | 3 | 2 | 3 | 3 | 2 | 3 | 3 | 2 | 3 |
| Mean | 3.8 | 2.1 | 3.2 | 4.4 | 2.8 | 4.8 | 4.6 | 2.9 | 4.9 |
| S.E. | 0.4 | 0.1 | 0.4 | 0.5 | 0.4 | 0.4 | 0.5 | 0.4 | 0.5 |
| S.D. | 1.2 | 0.3 | 1.1 | 1.4 | 1.2 | 1.4 | 1.5 | 1.3 | 1.5 |

| | |
|---|---|
| Friedman: | 1^{st} h P < 0.001 (significant), 2^{nd} h P < 0.001 (significant), |
| | 3^{rd} h P < 0.001 (significant) |
| Wilcoxon (P=): | one-sided/two-sided: (one-sided: significant, two-sided: |
| | significant) |
| | vs. pre-drug: 1^{st} h 0.002/0.004, 2^{nd} h 0.004/0.008, 3^{rd} h |
| | 0.004/0.008 |
| | vs. post-drug: 1^{st} h 0.008/0.016, 2^{nd} h 0.004/0.008, 3^{rd} h |
| | 0.004/0.008 |

6B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt 205/2 | 7 | 2 | 1 | 82 | — | 98 |
| dt 205/7 | 4 | 6 | 9 | 53 | 104 | 80 |
| dt 205/9 | 6 | 14 | 1 | 57 | — | 89 |
| dt 206/2 | 4 | 11 | 3 | — | — | — |
| dt 206/7 | 6 | 13 | 2 | 91 | — | 58 |
| dt 206/8 | 2 | 10 | 4 | 123 | — | 114 |
| dt 206/9 | 1 | 6 | 3 | — | — | 140 |
| dt 206/10 | 3 | 10 | 2 | — | — | — |
| dt 207/2 | 5 | 23 | 2 | — | — | — |
| dt 207/13 | 2 | 1 | 4 | — | — | — |
| Mean | 4.0 | 9.6 | 3.1 | n.d. | n.d. | n.d. |
| S.E. | 0.6 | 2.0 | 0.7 | n.d. | n.d. | n.d. |
| S.D. | 2.0 | 6.4 | 2.3 | n.d. | n.d. | n.d. |

| | | |
|---|---|---|
| Latency on: | Friedman: | P = 0.061 (not significant) |
| | Wilcoxon: | vs. pre-drug: one-sided P = 0.007, two-sided |
| | | P = 0.014 |
| | | vs. post-drug: one-sided P = 0.014, |
| | | two-sided P = 0.027 |
| | | (not significant because of Friedman) |

Latency max: not determined (n.d.) because less than 5 animals reached
stage 6 during drug and control trials

TABLE 7
Flupirtine 10 mg/kg (soluted in 0.9% NaCl) i.p., age: 32-38 days
of life
7A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 180/1 | 2 | 3 | 3 | 3 | 4 | 3 | 3 | 4 | 3 |
| dt 180/2 | 4 | 3 | 3 | 4 | 3 | 3 | 4 | 3 | 3 |
| dt 180/3 | 4 | 3 | 3 | 4 | 3 | 4 | 6 | 3 | 6 |
| dt 180/5 | 3 | 3 | 4 | 3 | 4 | 4 | 3 | 4 | 4 |
| dt 180/7 | 3 | 3 | 3 | 3 | 3 | 3 | 3 | 3 | 3 |
| dt 183/10 | 3 | 2 | 4 | 6 | 2 | 6 | 6 | 2 | 6 |
| dt 184/1 | 3 | 2 | 2 | 3 | 3 | 2 | 3 | 3 | 2 |
| dt 185/3 | 3 | 3 | 3 | 3 | 4 | 4 | 3 | 6 | 4 |
| Mean | 3.1 | 2.8 | 3.1 | 3.6 | 3.3 | 3.6 | 3.9 | 3.5 | 3.9 |
| S.E. | 0.2 | 0.2 | 0.2 | 0.4 | 0.3 | 0.4 | 0.5 | 0.4 | 0.5 |
| S.D. | 0.6 | 0.5 | 0.6 | 1.1 | 0.7 | 1.2 | 1.4 | 1.2 | 1.5 |

| | |
|---|---|
| Friedman (P=): | 1^{st} h 0.531, 2^{nd} h 1.00, 3^{rd} h 0.967 |
| | (not significant) |
| Wilcoxon (P=): | one-sided/two-sided: (one-sided: not significant, |
| | two-sided: not significant) |
| | vs. pre-drug: 1^{st} h 0.157/0.313, 2^{nd} h 0.422/0.844, 3^{rd} h |
| | 0.344/0.688 |
| | vs. post-drug: 1^{st} h 0.25/0.5, 2^{nd} h 0.438/0.875, 3^{rd} h |
| | 0.407/0.813 |

7B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt 180/1 | 7 | 4 | 4 | — | — | — |
| dt 180/2 | 4 | 8 | 8 | — | — | — |
| dt 180/3 | 8 | 9 | 6 | 163 | — | 125 |
| dt 180/5 | 3 | 3 | 2 | — | — | — |
| dt 180/7 | 5 | 5 | 8 | — | — | — |
| dt 183/10 | 8 | 14 | 2 | 115 | — | 116 |
| dt 184/1 | 7 | 1 | 3 | — | — | — |
| dt 185/3 | 6 | 11 | 4 | — | — | — |
| Mean | 6.0 | 6.9 | 4.6 | n.d. | n.d. | n.d. |
| S.E. | 0.7 | 1.6 | 0.9 | n.d. | n.d. | n.d. |
| S.D. | 1.9 | 4.4 | 2.5 | n.d. | n.d. | n.d. |

| | | |
|---|---|---|
| Latency on: | Friedmann: | P = 0.531 (not significant) |
| | Wilcoxon: | vs. pre-drug: one-sided P = 0.282, two-sided |
| | | P = 0.563 |
| | | vs. post-drug: one-sided P = 0.157, |
| | | two-sided P = 0.313 |
| | | (one-sided: not significant) |

Latency max: not determined (n.d.) because less than 5 animals reached
stage 6 during drug and control trials

TABLE 8
Flupirtine: 20 mg/kg (soluted in 0.9% NaCl) i.p.; age: 36-38
days of life
8A. Severity (score) of dystonia

| | Maximum | | | Maximum | | | Maximum | | |
|---|---|---|---|---|---|---|---|---|---|
| Animal | severity 1^{st} h | | | severity 2^{nd} h | | | severity 3^{rd} h | | |
| (number) | pre | drug | post | pre | drug | post | pre | drug | post |
| dt 194/1 | 2 | 2 | 3 | 3 | 2 | 3 | 3 | 2 | 3 |
| dt 194/3 | 2 | 2 | 5 | 4 | 2 | 6 | 6 | 2 | 6 |
| dt 194/4 | 3 | 2 | 3 | 3 | 4 | 4 | 4 | 4 | 6 |
| dt 195/2 | 3 | 2 | 3 | 3 | 2 | 3 | 3 | 2 | 3 |
| dt 195/3 | 3 | 2 | 3 | 5 | 2 | 3 | 6 | 2 | 4 |
| dt 199/1 | 3 | 2 | 3 | 3 | 3 | 3 | 4 | 3 | 4 |
| dt 199/2 | 6 | 2 | 5 | 6 | 5 | 6 | 6 | 6 | 6 |
| dt 199/4 | 2 | 2 | 4 | 4 | 2 | 6 | 4 | 3 | 6 |
| dt 199/6 | 4 | 2 | 3 | 4 | 2 | 6 | 4 | 3 | 6 |
| dt 199/10 | 2 | 2 | 4 | 3 | 2 | 4 | 6 | 2 | 4 |
| Mean | 3.0 | 2.0 | 3.6 | 3.8 | 2.6 | 4.4 | 4.6 | 2.9 | 4.8 |
| S.E. | 0.4 | 0 | 0.3 | 0.3 | 0.3 | 0.5 | 0.4 | 0.4 | 0.4 |
| S.D. | 1.3 | 0 | 0.8 | 1.0 | 1.1 | 1.4 | 1.3 | 1.3 | 1.3 |

| | |
|---|---|
| Friedman: | 1^{st} h P = 0.002, 2^{nd} h P = 0.003, 3^{rd} h P < 0.001 |
| | (significant) |
| Wilcoxon (P=): | one-sided/two-sided: (one-sided: significant, two-sided: |
| | significant) |
| | vs. pre-drug: 1^{st} h 0.016/0.031, 2^{nd} h 0.01/0.02, 3^{rd} h |
| | 0.004/0.008 |
| | vs. post-drug: 1^{st} h 0.001/0.002, 2^{nd} h 0.004/0.008, 3^{rd} h |
| | 0.004/0.002 |

8B. Latency onset (latency on stage 2) and latency max (latency to
stage 6)

| | Latency | | | Latency | | |
|---|---|---|---|---|---|---|
| Animal | onset (min) | | | max (min) | | |
| (number) | pre | drug | post | pre | drug | post |
| dt 194/1 | 1 | 2 | 4 | — | — | — |
| dt 194/3 | 2 | 1 | 4 | 133 | — | 130 |
| dt 194/4 | 0 | 3 | 2 | — | — | 158 |
| dt 195/2 | 3 | 18 | 6 | — | — | — |
| dt 195/3 | 2 | 23 | 4 | 131 | — | — |
| dt 199/1 | 3 | 2 | 6 | — | — | — |
| dt 199/2 | 1 | 13 | 10 | 56 | 154 | 139 |
| dt 199/4 | 3 | 37 | 2 | — | — | 92 |
| dt 199/6 | 2 | 25 | 2 | — | — | 89 |
| dt 199/10 | 1 | 14 | 3 | 141 | — | — |
| Mean | 1.8 | 13.8 | 4.1 | n.d. | n.d. | n.d. |
| S.E. | 0.3 | 3.8 | 0.8 | n.d. | n.d. | n.d. |
| S.D. | 1.0 | 12.1 | 2.6 | n.d. | n.d. | n.d. |

| | | |
|---|---|---|
| Latency on: | Friedmann: | P = 0.038 (significant) |
| | Wilcoxon: | vs. pre-drug: one-sided P = 0.007, two-sided |
| | | P = 0.014 (significant) |
| | | vs. post-drug: one-sided P = 0.042 |
| | | (significant), two-sided P = 0.084 |
| | | (not significant) |

Latency max: not determined (n.d.) because less than 5 animals reached
stage 6 during drug and control trials

### EXAMPLE 2

### Effects of the Kᵥ7.2/7.3 Channel Blocker XE-991 in the dt^{sz} Mutant Hamster, a Model of Paroxysmal Dystonia

Aim: With regard to the antidystonic effects of the potassium channel openers retigabine and flupirtine (see Example 1) we examined the effects of the selective Kᵥ7.2/7.3 channel blocker XE-991 (10,10-bis(4-pyridinylmethyl)-9(10H)-anthracenone) on the severity of dystonic episodes in the dt^{sz} mutant hamster. Furthermore, we investigated if the antidystonic effects of retigabine can be counteracted by combined treatment with XE-991.

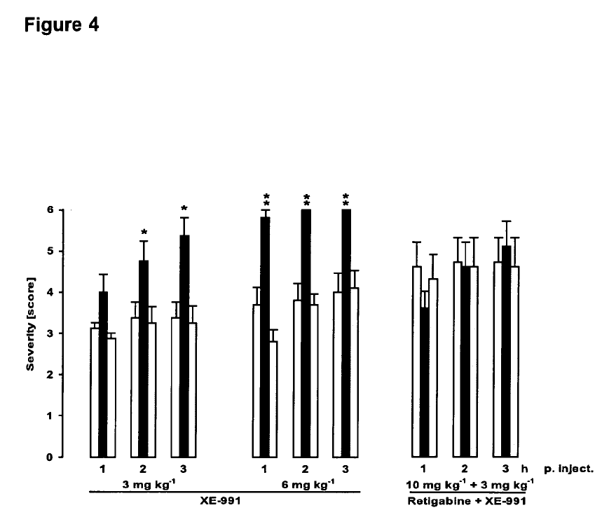

Results: The Kᵥ7.2/7.3 channel blocker XE-991 caused an aggravation of dystonia, i.e., increased the maximum severity of dystonia at doses of 3 and 6 mg/kg i.p. (FIG. 4). The latency to onset of dystonia tended to be decreased after treatment with XE-991 (not illustrated). Two out of 8 animals exhibited moderate to unequivocal hyperlocomotion and moderate ataxia (up to 180 min) and marked initial facial contortions 10-20 min after administration of 6 mg/kg. All hamsters which were treated with the higher dose exhibited facial contortions, salivation and increased defecation. Furthermore, unequivocal hyperlocomotion and ataxia were observed during the first h after administration. As shown in FIG. 4, pretreatment with XE-991 (3 mg/kg i.p.) 10 min prior administration of retigabine (10 mg/kg i.p.) counteracted the antidystonic effect of retigabine.

Conclusions: The present results clearly indicate that the antidystonic effect of retigabine is mediated by activation of Kᵥ7.2/7.3 channels. These data support our suggestion that dysfunctions of Kᵥ7.2/7.3 channels deserve attention in the research of the pathophysiology of dystonias and dystonia-associated dyskinesias and that Kᵥ7.2/7.3 channel activators are interesting compounds for the treatment of these movement disorders.

FIG. 4. Effect of the KCNQ channel blocker XE-991 on severity of dystonia in mutant hamsters after intraperitoneal injections of 3 and 6 mg/kg alone or of 3 mg/kg 10 min after administration of retigabine (10 mg/kg i.p.). The white bars in each set of three bars indicate the control values obtained two days before drug administration (pre drug control). The black bar refers to the day of drug administration in the same animal groups. The gray bars in each set of three bars indicate the control values obtained two days after drug administration (post drug control). The figure shows the average of the maximum individual severity scores of dystonia reached within the 1st, 2nd and 3rd h post injection of isotonic saline (control trials) or of XE-991, reflecting the progression of dystonia in dt^{sz} hamsters during control recordings and after treatment with the active compound. Asterisks indicate significant increase of the severity of dystonia in comparison to the pre- and post-drug control (*P<0.05, **P<0.01). Data are shown as means ±S.E.

### EXAMPLE 3

### Effect of Flupirtine and Retigabine as Examples for Neuronal Potassium Channel Activators in a Chronic Model of L-DOPA Induced Dyskinesia

Rational: The idiopathic Parkinson syndrome is a common neurodegenerative disease, in which a progressive degeneration of dopaminergic neurons in the substantia nigra leads to decreased striatal dopamine levels. Considering the patient profile, levodopa in combination with decarboxylase-inhibitors (e.g. benserazide) represents still the therapeutical “gold standard” in many cases. However, many patients develop dyskinesias after long-term treatment. The pathophysiology of these spontaneous involuntary dystonic and choreatic movements is unclear, but an increased activity of striatal projection neurons seems to play a critical role. These neurons express Kv7 channels i.e. one type of neuronal potassium channels, which cause a hyperpolarization after voltage-dependent activation. Based on previous observations in a mutant hamster model of paroxysmal dystonia, it was concluded that neuronal potassium channel openers and especially flupirtine and retigabine are capable of reducing symptoms of dystonia. This model has been discussed to be predictive also for other forms of dystonia, i.e. L-DOPA induced dyskinesia and neuroleptics induced dyskinesia, myokymia and neuromyotonia. The data have been summarized in example 1 and 2. As the neuronal potassium channel openers retigabine and flupirtine proved to be antidystonic in an animal model of paroxysmal dystonia, we verified in a more disease related model the results obtained in the hamster model. The selected model is a model of L-DOPA induced dyskinesial. To verify that neuronal potassium channel activators indeed are a new treatment option for such drug-induced dyskinesias, the model of L-DOPA induced dyskinesia was established at the Department of Pharmacology of the Free University of Berlin (Prof. A. Richter).

Method: The model has been developed by Cenci, Lee and Bjorklund (L-DOPA-induced dyskinesia in the rat is associated with striatal overexpression of prodynorphin- and glutamic acid decarboxylase mRNA, Eur J. Neurosci. 1998; 10:2694-706) and was modified to show more severe dyskinesia. In brief, rats are first unilaterally lesioned in the medial forebrain bundle resulting in a near complete loss of the mesostriatal dopamine pathway. After a recovery, rats receive daily doses of both, L-DOPA and benserazide. Within less than 3 weeks, rats develop typical dyskinetic movements, both affecting their forearms and the oral regions. These dyskinetic movements are chronic and re-occur upon LDOPA challenge even after a treatment holiday of several weeks indicating that a chronic change has occurred. The dyskinesia can be quantified using an easy and reliable scoring system developed by Cenci et al.

Dopamine-denervating lesions were performed by unilateral injections of 8 μg 6-OHDA in the left medial forebrain bundle of female Sprague-Dawley rats. All rats were tested for amphetamine-induced rotational behaviour 2 weeks after the injection. Rats showing >4 ipsilateral rotations/min over a 90-minute period were presumed to have a striatal dopamine depletion of more than 90%. At 4 weeks post lesion, 2 groups started to receive chronic treatment with either 20 mg/kg levodopa and 15 mg/kg benserazide or vehicle for 20 days. For rating, dyskinesia was classified into three subtypes (limb, axial and orolingual) and scored from 0 (=absent) to 4 (=permanent, not suppressible) over 200 minutes and every 30 minutes.

Results and Discussion: For drug testing, retigabine (2.5 and 5 mg/kg) was administered additional to levodopa and vehicle respectively. Effects of drug action in comparison to vehicle controls were detected by adding up the severity scores of each observation time. Retigabine reduced the severity of dyskinesia significantly from the 110^{th} to 140^{th} minute of observation after intraperitoneal (i.p.) administration of 2.5 mg/kg (p<0.05) and 50, 80 (each p<0.05) and 110 (p<0.01) minutes after i.p. injection of 5 mg/kg. The higher dosage of retigabine caused marked hypolocomotion and ataxia during the first hour of observation.

To test the effect of flupirtine, the compound was administered at the dose of 10 mg/kg i.p. after administration of L-DOPA in these rats. A full cross over design was utilized to account for individual variability. As expected, flupirtine was also capable of suppressing the typical dyskinetic symptoms and was also well tolerated. The studies are ongoing and will we extended to cover a full dose range. Also, other neuronal potassium channel openers covered by the patent application will be tested. These data already indicate that flupirtine comprises a unique opportunity to treat late stage Parkinson's patients with L-DOPA induced dyskinesia. Since flupirtine has been shown to also potentiate the activity of L-DOPA, addition of flupirtine to the primary medication can be expected to improve the over all treatment effect in these patients. The results of our study suggest that openers of KV7 channels and more generally of neuronal potassium channels are interesting candidates for the treatment of levodopa-induced dyskinesia. Both, retigabine and flupirtine, are known to be well tolerated by patients. Additionally, dyskinetic patients, who often suffer from painful muscle distortions, could benefit from the analgesic actions of these compounds. Based on the mechanism of action it is likely that the compounds not only reduce the symptoms but also are able to delay the development of levodopa-induced dyskinesias.

1. A method for the treatment, inhibition, or prevention of a movement disorder comprising administering to a mammal in need thereof a therapeutically effective amount of a neuronal potassium channel opener or a pharmacologically acceptable derivative thereof.

2. The method according to claim 1, wherein the movement disorder is selected from primary dystonia, paroxysmal dystonia, secondary dystonia, drug induced dystonia/dyskinesia, tardive dystonia, neuroleptics induced dystonia, treatment induced dystonia/dyskinesia in Parkinson's disease patients, heredodegenerative dystonia, dystonia in Huntington's disease patients, dystonia in Tourette's syndrome patients, dystonia in Restless Leg syndrome patients, dystonia like symptoms in patients with Tics, dystonia-associated dyskinesias, paroxysmal dyskinesias, paroxysmal non-kinesigenic dyskinesia, paroxysmal dystonic choreoathetosis, paroxysmal kinesigenic dyskinesia, paroxysmal kinesigenic choreoathetosis, the exertion-induced dyskinesia, hypnogenic paroxysmal dyskinesia, drug-induced dyskinesia, myokymia, and neuromyotonia.

3. The method according to claim 1, wherein the neuronal potassium channel opener comprises flupirtine (ethyl-N-[2-amino-6-(4-fluorophenylmethylamino)pyridin-3-yl]-carbamate), or a pharmacologically acceptable derivative thereof.

4. The method according to claim 1, wherein the neuronal potassium channel opener comprises retigabine (2-amino-4-(4-fluorobenzylamino)-1-ethoxycarbonylaminobenzene) or a pharmacologically acceptable derivative thereof.

5. The method according to claim 1, wherein the neuronal potassium channel opener comprises Maxipost (BMS 204352), (3-(5-chloro-2-methoxyphenyl)-1,3-dihydro-3-fluoro-6-(trifluoromethyl)-2H-indole-2-one), an enantiomer thereof, or a pharmacologically acceptable derivative thereof.

6. The method of according to claim 1, wherein the mammal is a human.

7. The method according to claim 1, wherein the mammal is at least one of a cat or a dog.

8. The method of claim 3, wherein the flupirtine is administered at a daily dose of between 200 and 1800 mg per day, and wherein the daily dose is calculated on the basis of the free base form of flupirtine.

9. The method of claim 4, wherein the retigabine is administered at a daily dose of between 200 and 1800 mg per day, and wherein the daily dose is calculated on the basis of the free base form of retigabine.

10. The method of claim 5, wherein the Maxipost is administered at a daily dose of between 10 and 600 mg per day.

11. The method of claim 1, wherein the administering is done orally, intravenously, rectally, parenterally, transdermally or via inhalation.

12. The method of claim 1, wherein the neuronal potassium channel opener is a pharmacologically acceptable salt or amide thereof.

13. The use method of claim 1, further comprising administering an additional therapeutic agent for the treatment of an underlying disease of the movement disorder.

14. The method of claim 13, wherein the underlying disease is selected from the group consisting of Wilson disease, multiple sclerosis, and Parkinson's disease.
